SEQUENCE LISTING (43 sequences)

SEQ ID NO: 1 - protein, 5 aa, Artificial Sequence
  Description of Artificial Sequence Synthetic peptide
EYYMY

SEQ ID NO: 2 - protein, 17 aa, Artificial Sequence
  Description of Artificial Sequence Synthetic peptide
RIDPEDGSIDYVEKFKK

SEQ ID NO: 3 - protein, 9 aa, Artificial Sequence
  Description of Artificial Sequence Synthetic peptide
GKFNYRFAY

SEQ ID NO: 4 - protein, 16 aa, Artificial Sequence
  Description of Artificial Sequence Synthetic peptide
RSSQSLLHSSGNTYLN

SEQ ID NO: 5 - protein, 7 aa, Artificial Sequence
  Description of Artificial Sequence Synthetic peptide
LVSKLES

SEQ ID NO: 6 - protein, 9 aa, Artificial Sequence
  Description of Artificial Sequence Synthetic peptide
MQFTHYPYT

SEQ ID NO: 7 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 8 - protein, 46 aa, Artificial Sequence
  Description of Artificial Sequence Synthetic polypeptide
AKPTTTPAPRPPTPAPTIASQPLSLRPEACRPAAGGAVHTRGLDFA

SEQ ID NO: 9 - protein, 88 aa, Artificial Sequence
  Description of Artificial Sequence Synthetic polypeptide
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 10 - protein, 28 aa, Artificial Sequence
  Description of Artificial Sequence Synthetic peptide
LDPKLCYLLDGILFIYGVILTALFLRVK

SEQ ID NO: 11 - protein, 21 aa, Artificial Sequence
  Description of Artificial Sequence Synthetic peptide
LCYLLDGILFIYGVILTALFL

SEQ ID NO: 12 - protein, 27 aa, Artificial Sequence
  Description of Artificial Sequence Synthetic peptide
FWVLVVVGGVLACYSLLVTVAFIIFWV

SEQ ID NO: 13 - protein, 66 aa, Artificial Sequence
  Description of Artificial Sequence Synthetic polypeptide
IEVMYPPPYLDNEKSNGTIIHVKGKHLCPSPLFPGPSKPFWVLVVVGGVLACYSLLVTVAFIIFWV

SEQ ID NO: 14 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 16 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 17 - protein, 5 aa, Artificial Sequence
  Description of Artificial Sequence Synthetic peptide
GGGGS

SEQ ID NO: 18 - protein, 15 aa, Artificial Sequence
  Description of Artificial Sequence Synthetic peptide
GGGGSGGGGSGGGGS

SEQ ID NO: 19 - protein, 5 aa, Artificial Sequence
  Description of Artificial Sequence Synthetic peptide
EYYMY

SEQ ID NO: 20 - protein, 17 aa, Artificial Sequence
  Description of Artificial Sequence Synthetic peptide
RIDPEDGSIDYVEKFKK

SEQ ID NO: 21 - protein, 9 aa, Artificial Sequence
  Description of Artificial Sequence Synthetic peptide
GKFNYRFAY

SEQ ID NO: 22 - protein, 16 aa, Artificial Sequence
  Description of Artificial Sequence Synthetic peptide
RSSQSLLHSSGNTYLN

SEQ ID NO: 23 - protein, 7 aa, Artificial Sequence
  Description of Artificial Sequence Synthetic peptide
LVSKLES

SEQ ID NO: 24 - protein, 9 aa, Artificial Sequence
  Description of Artificial Sequence Synthetic peptide
MQFTHYPYT

SEQ ID NO: 25 - protein, 118 aa, Artificial Sequence
  Description of Artificial Sequence Synthetic polypeptide
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 26 - protein, 112 aa, Artificial Sequence
  Description of Artificial Sequence Synthetic polypeptide
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 27 - protein, 118 aa, Artificial Sequence
  Description of Artificial Sequence Synthetic polypeptide
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 28 - protein, 112 aa, Artificial Sequence; identical to SEQ ID NO: 26
  Description of Artificial Sequence Synthetic polypeptide

SEQ ID NO: 29 - protein, 120 aa, Artificial Sequence
  Description of Artificial Sequence Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 30 - protein, 109 aa, Artificial Sequence
  Description of Artificial Sequence Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 31 - protein, 351 aa, Homo sapiens; identical to SEQ ID NO: 7

SEQ ID NO: 32 - protein, 7 aa, Artificial Sequence
  Description of Artificial Sequence Synthetic peptide
GFTFSSY

SEQ ID NO: 33 - protein, 5 aa, Artificial Sequence
  Description of Artificial Sequence Synthetic peptide
SGGGF

SEQ ID NO: 34 - protein, 9 aa, Artificial Sequence
  Description of Artificial Sequence Synthetic peptide
SSYGEIMDY

SEQ ID NO: 35 - protein, 9 aa, Artificial Sequence
  Description of Artificial Sequence Synthetic peptide
SSYGELMDY

SEQ ID NO: 36 - protein, 11 aa, Artificial Sequence
  Description of Artificial Sequence Synthetic peptide
RASQRIGTSIH

SEQ ID NO: 37 - protein, 7 aa, Artificial Sequence
  Description of Artificial Sequence Synthetic peptide
YASESIS

SEQ ID NO: 38 - protein, 10 aa, Artificial Sequence
  Description of Artificial Sequence Synthetic peptide
QQSHGWPFTF

SEQ ID NO: 39 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSS

SEQ ID NO: 40 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGELMDYWGQGTSVTVSS

SEQ ID NO: 41 - protein, 107 aa, Artificial Sequence
  Description of Artificial Sequence Synthetic polypeptide
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIE

SEQ ID NO: 42 - protein, 324 aa, Artificial Sequence
  Description of Artificial Sequence Synthetic polypeptide
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 107 aa, Artificial Sequence
  Description of Artificial Sequence Synthetic polypeptide
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2021/0260212 A1
(43) Pub. Date: Aug. 26, 2021

(54) USE OF AN ANTI-CD2 ANTIBODY DRUG CONJUGATE (ADC) IN ALLOGENEIC CELL THERAPY

(71) Applicant: Magenta Therapeutics, Inc., Cambridge, MA (US)

(72) Inventors: Anthony Boitano, Newton, MA (US); Michael Cooke, Boston, MA (US)

(21) Appl. No.: 17/155,721

(22) Filed: Jan. 22, 2021

Related U.S. Application Data
(63) Continuation of application No. PCT/US2019/043122, filed on Jul. 23, 2019.
(60) Provisional application No. 62/773,108, filed on Nov. 29, 2018.
(60) Provisional application No. 62/702,301, filed on Jul. 23, 2018.

(51) Int. Cl. A61K 47/68; C07K 16/28; A61P 43/00; C07K 14/705

(52) U.S. Cl. CPC A61K 47/6843; C07K 14/705; A61P 43/00; C07K 16/2806

(57) ABSTRACT

The invention provides methods of depleting CD2+ cells in human patients undergoing chimeric antigen receptor (CAR) immunotherapy in order to promote acceptance of CAR expressing immune cells. Anti-CD2 antibody drug conjugates (ADCs) are administered as a conditioning regimen to a human patient receiving autologous or allogeneic CAR expressing immune cells such that the CAR expressing immune cells are accepted by the human patient. Compositions and methods of the invention can be used in combination with CAR therapy to treat a variety of pathologies, including autoimmune diseases and cancer.

### RELATED APPLICATIONS

This application is a continuation of PCT Application No. PCT/US2019/043122, filed on Jul. 23, 2019, which claims priority to U.S. Provisional Application No. 62/702,301, filed on Jul. 23, 2018, and U.S. Provisional Application No. 62/773,108, filed on Nov. 29, 2018. The entire contents of the foregoing applications are hereby incorporated by reference.

### SEQUENCE LISTING

The instant application contains a Sequence Listing which has been submitted electronically in ASCII format and is hereby incorporated by reference in its entirety. Said ASCII copy, created on Jan. 21, 2021, is named M103034_2050US.C1_SL.txt and is 30,866 bytes in size.

### FIELD OF THE INVENTION

The present invention generally relates to methods for promoting acceptance of an immune cell expressing a chimeric antigen receptor (CAR) in a human subject through the use of an anti-CD2 antibody-drug conjugate (ADC).

### BACKGROUND OF THE INVENTION

Chimeric antigen receptor (CAR) therapy is an immunological treatment that uses the body's own immune system to destroy cells expressing a specific antigen associated with a certain disease, such as cancer. In cancer, for example, CAR therapy enlists and strengthens the power of a patient's immune system to attack tumors. Over the past several years, this immunotherapy has emerged as a promising and revolutionary therapy. CAR therapy is based on an immune cell, such as a T cell, expressing a CAR which is generally a transmembrane fusion protein that combines an extracellular antigen binding domain, such as an scFv, with cytoplasmic activity signaling and “co-stimulatory” domains that signal the cell from the surface receptor. Thus, when immune cells, such as T-cells, express CARs, the immune cells are able to recognize and kill cells that express the antigen targeted by the antigen binding domain of the CAR (e.g., a tumor associated antigen) (Geyer and Brentjens (2016) Cytotherapy 18(11): 1393-1409).

While CAR therapy is an incredibly powerful technology, it does come with serious risks and adverse side effects (Kay and Turtle (2017) Drugs 77(3):237-245; Hill et al. (2018) Blood 131:121-130). Lymphodepleting chemotherapy is commonly used as a conditioning treatment in combination with CAR therapy in order to minimize the rejection of the CAR expressing cells by the patient receiving treatment (Wei et al. (2017) Exp Hematol Oncol. 6: 10). For example, the combination of lymphodepleting agents fludarabine and cyclophosphamide improved duration of CAR-T cells in recipient patients (Turtle et al. (2016) J Clinic Invest 126(6):2123; see also US 20170368101). While conditioning therapy has improved the efficacy of CAR-T cells, lymphodepleting chemotherapy often has serious negative side effects.

### SUMMARY OF THE INVENTION

The present invention provides a conditioning regimen which can be used with CAR therapy to promote acceptance of CAR expressing immune cells. The methods described herein can be used to promote acceptance of either autologous CAR expressing immune cells or allogeneic CAR expressing immune cells. Traditionally acceptance of such cells has been achieved using lymphodepleting chemotherapeutic treatment. Described herein are improved methods of promoting acceptance of CAR expressing cells in a recipient patient.

In a first aspect, the invention features a method of promoting acceptance of an immune cell expressing a chimeric antigen receptor (CAR) in a human subject having cancer or an autoimmune disease, by (a) administering an anti-CD2 antibody drug conjugate (ADC) to a human subject having cancer or an autoimmune disease, wherein the anti-CD2 ADC comprises an anti-CD2 antibody, or antigen-binding fragment thereof, conjugated to a cytotoxin via a linker; and (b) administering a therapeutically effective amount of an immune cell expressing a CAR to the human subject, wherein the CAR comprises an extracellular domain that binds to a tumor antigen or an antigen associated with an autoimmune disease, a transmembrane domain, and a cytoplasmic domain. In one embodiment, the human subject is not administered alemtuzumab prior to, concomitantly with, or following step (b). In another embodiment, the human subject is not administered a lymphodepleting chemotherapeutic agent prior to, concomitantly with, or following step (b). In yet other embodiments, the lymphodepleting chemotherapeutic agent is fludarabine, cyclophosphamide, bendamustine, and/or pentostatin.

In certain embodiments, the method involves administering an anti-CD2 ADC to the human subject prior to step (b).

In certain other embodiments, method involves administering the anti-CD2 ADC to the human subject about 12 hours to about 21 days before step (b).

In certain embodiments, the immune cell is an allogeneic cell or an autologous cell. In yet another embodiment, the allogeneic cell is an allogeneic T cell or an allogeneic NK cell.

In certain embodiments, the therapeutically effective amount of the allogeneic cell expressing the CAR is about 1×10⁴ to about 1.0×10⁸ cells/kg.

In another aspect, the invention features a method of treating a patient having a tumor by administering (i) an anti-CD2 ADC, wherein the anti-CD2 ADC comprises an anti-CD2 antibody, or antigen-binding fragment thereof, conjugated to a cytotoxin via a linker, and (ii) administering to the patient a therapeutically effective amount of from about 1×10⁶ to about 1×10⁸ engineered CAR T cells/kg. In one embodiment, the therapeutically effective amount of the engineered CAR T cells is about 1×10⁶ or about 2×10⁶ cells/kg. In yet another embodiment, the anti-CD2 ADC is administered to the patient as a single dose or as multiple doses.

In certain embodiments, the anti-CD2 antibody, or antigen-binding fragment thereof, comprises a heavy chain variable region comprising a CDR1, a CDR2, and a CDR3 having an amino acid sequence as set forth in SEQ ID NOs: 1, 2, and 3, respectively, and comprises a light chain variable region comprising a CDR1, a CDR2, and a CDR3 having an amino acid sequence as set forth in SEQ ID NOs: 4, 5, and 6, respectively. In another embodiment, the anti-CD2 antibody, or antigen-binding fragment thereof, is chimeric or humanized.

In certain embodiments, the anti-CD2 antibody, or antigen-binding fragment thereof, is an IgG1 isotype or an IgG4 isotype.

In certain embodiments, the cytotoxin is an antimitotic agent or an RNA polymerase inhibitor. In other embodiments, the cytotoxin is a maytansine, a calicheamicin, a pyrrolobenzodiazepine, an indolinobenzodiazepine, or an auristatin. In one embodiment, the auristatin is monomethyl auristatin F (MMAF) or monomethyl auristatin E (MMAE). In one embodiment, the cytotoxin is a maytansine. In one embodiment, the cytotoxin is a pyrrolobenzodiazepine (PBD). For example, in some embodiments, the PBD may selected from tesirine or talirine. In some embodiments, the cytotoxin may be a calicheamicin. For example, in some embodiments, the calicheamicin may be ozogamicin.

In other embodiments, the RNA polymerase inhibitor is an amatoxin. In another embodiment, the RNA polymerase inhibitor is an amanitin. In another embodiment, the amanitin is selected from the group consisting of α-amanitin, β-amanitin, γ-amanitin, ε-amanitin, amanin, amaninamide, amanullin, amanullinic acid, and proamanullin.

In one embodiment, the antibody-drug conjugate (ADC) is represented by the formula Ab-Z-L-Am, wherein Ab is an antibody or antigen-binding fragment thereof that binds CD2, L is a linker, Z is a chemical moiety, and Am is an amatoxin. In certain embodiments, the linker-amatoxin conjugate Am-L-Z is represented by formula (III)

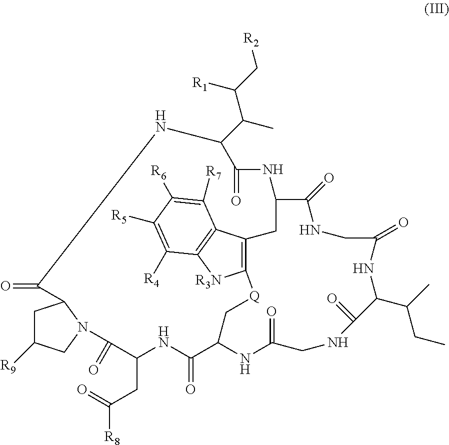

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄, R₅, R₆, and R₇ are each independently H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

Q is —S—, —S(O)—, or —SO₂—

R_{C} is -L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;

L is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; or comprises a dipeptide; or —((CH₂)ₘO)ₙ(CH₂)ₘ—, where m and n are each independently selected from 1, 2, 3, 4, 5, 6, 7, 8, 9, and 10; and

Z is a chemical moiety formed from a coupling reaction between a reactive substituent present on L and a reactive substituent present within the anti-CD2 antibody or antigen-binding fragment thereof.

In certain embodiments, the linker-amatoxin conjugate Am-L-Z is represented by formula (III)

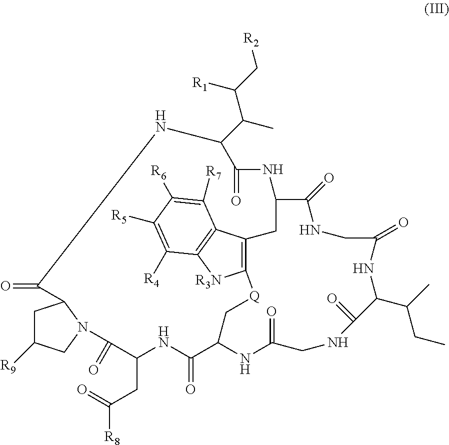

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄, R₅, R₆, and R₇ are each independently H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

Q is —S—, —S(O)—, or —SO₂—

R_{C} is -L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;

L is a linker; and

Z is a chemical moiety formed from a coupling reaction between a reactive substituent present on L and a reactive substituent present within the anti-CD2 antibody or antigen-binding fragment thereof.

In one embodiment of Formula (III), L is a peptide containing linker.

In some embodiments, the linker comprises one or more of a dipeptide, a p-aminobenzyl (PAB) group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, —(C═O)—, a —(CH₂CH₂O)ₚ— group, wherein p is an integer from 1-6, ((CH2)ₘO)ₙ(CH₂)ₘ—, where n and each m are each independently selected from 1, 2, 3, 4, 5, 6, 7, 8, 9, and 10; or a combination thereof.

In some embodiments, the linker comprises a ((CH₂)ₘO)ₙ(CH₂)ₘ— group and a heteroaryl group, wherein the heteroaryl group is a triazole. In some embodiments, the ((CH₂)ₘO)ₙ(CH₂)ₘ—group and triazole together comprise

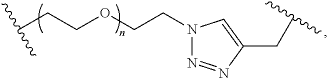

where n is from 1 to 10, and the wavy lines indicate attachment points to additional linker components, the chemical moiety Z, or the amatoxin.

In some embodiments, Am contains exactly one R_{C} substituent.

In one embodiment, the linker of the ADC is N-beta-maleimidopropionyl-Val-Ala-para-aminobenzyl (BMP-Val-Ala-PAB). In some embodiments, the linker L and the chemical moiety Z, taken together as L-Z, is

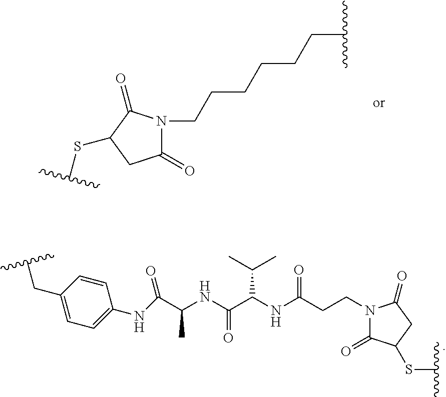

where S is a sulfur atom which represents the reactive substituent present within an antibody, or antigen-binding fragment thereof, that binds CD2 (e.g., from the —SH group of a cysteine residue).

In some embodiments, L-Z is

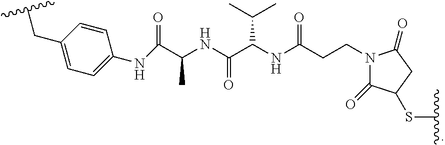

In one embodiment, the ADC is represented by any one of the following structures:

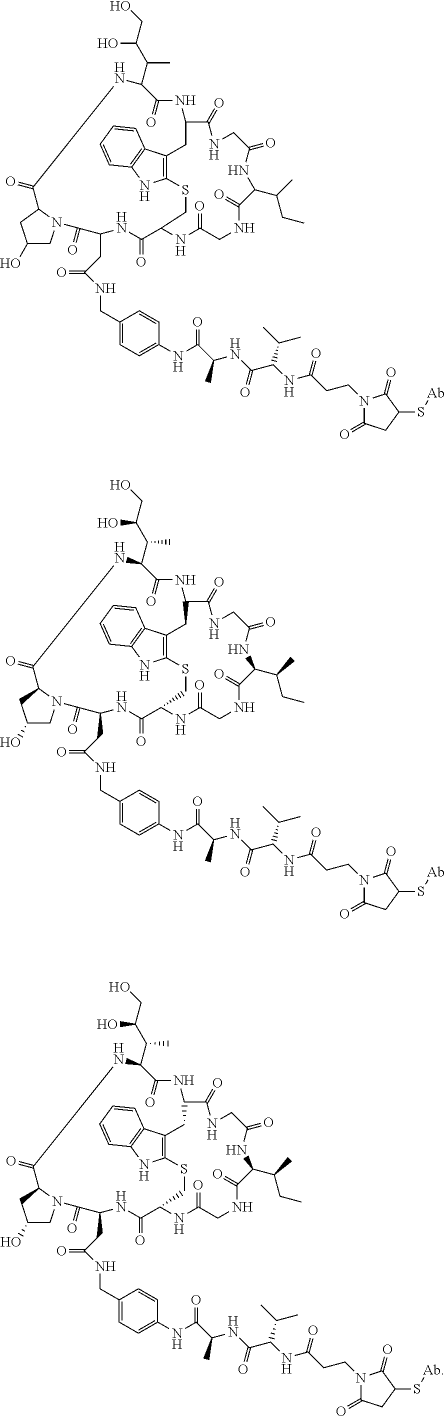

In one embodiment, the ADC is represented by one of the following structures:

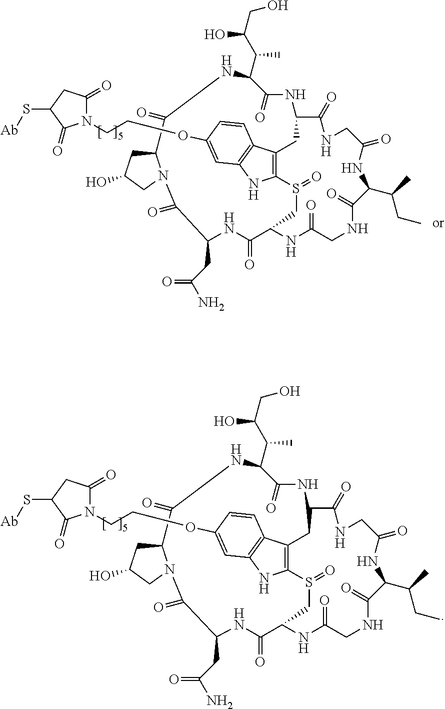

In certain embodiments, the ADC has a serum half-life of 3 days or less.

In certain embodiments, the extracellular domain of the CAR comprises an scFv antibody or a single chain T cell receptor (scTCR).

In certain embodiments, the extracellular domain comprises a non-immunoglobulin scaffold protein.

In certain embodiments, the tumor antigen is an antigen selected from the group consisting of CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

In certain embodiments, the cytoplasmic domain of the CAR comprises a CD28 cytoplasmic signaling domain, a CD3 zeta cytoplasmic signaling domain, an OX40 cytoplasmic signaling domain, and/or a CD137 (4-1BB) cytoplasmic signaling domain.

In certain embodiments, the cytoplasmic domain of the CAR comprises a CD3 zeta cytoplasmic signaling domain.

In certain embodiments, the human subject having cancer has a cancer selected from the group consisting of leukemia, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphoblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

In certain embodiments of any of the above aspects, the anti-CD2 antibody, or antigen-binding fragment thereof contains a combination of CDRs (i.e., CDR-H1, CDR-H2, CDR-H3, CDR-L1, CDR-L2, and CDR-L3 regions) as set forth in Table 4, below. In certain embodiments, the anti-CD2 antibody, or antigen-binding fragment thereof contains a combination of a heavy chain variable region and a light chain variable region as set forth in Table 4, below.

### BRIEF DESCRIPTION OF THE FIGURES

FIG. 1 graphically depicts the results of an in vitro cell line binding assay in which each of the indicated anti-CD2 antibodies or a negative control (i.e., mIgG1) was incubated with MOLT-4 cells (i.e., a human T lymphoblast cell line) followed by incubation of a fluorophore-conjugated anti-IgG antibody. Signal was detected through flow cytometry and is indicated as the geometric mean fluorescence intensity (y-axis) as a function of anti-CD2 antibody concentration (x-axis).

FIG. 2 graphically depicts the results of an in vitro primary cell binding assay in which the indicated anti-CD2 antibody (RPA-2.10 or “CD2 RPA-2.10”) or a negative control (i.e., mIgG1) was incubated with primary human T-cells followed by incubation of a fluorophore-conjugated anti-IgG antibody. Signal was detected through flow cytometry and is indicated as the geometric mean fluorescence intensity (y-axis) as a function of anti-CD2 antibody concentration (x-axis).

FIGS. 3A and 3B graphically depict results of an in vitro T cell killing assay including an anti-CD2-amanitin ADC (i.e., RPA-2.10-AM or “CD2 RPA-2.10-AM”) having an interchain conjugated amanitin with an average drug-to-antibody ratio of 6 (FIG. 3A) or a site-specific conjugated amanitin with a drug-to-antibody ratio of 2 (FIG. 3B). In FIG. 3A, the anti-CD2-ADC T-cell killing analysis is shown in comparison to an unconjugated anti-CD2 antibody (i.e., “CD2 RPA-2.10 NAKED”). In FIG. 3B, the anti-CD2-ADC T-cell killing analysis is shown in comparison to an anti-CD2 antibody having a H435A mutation (i.e., “CD2 RPA-2.10 D265C.H435A AM”) that decreases the half-life of the antibody. The results show the number of viable T-cells (y-axis) as a function of ADC (CD2 RPA-2.10 AM, CD2 RPA-2.10 D265C.H435A AM) or unconjugated antibody (CD2 RPA-2.10 NAKED) concentration (x-axis), as assessed using flow cytometry.

FIG. 4 graphically depicts results of an in vitro natural killer (NK) cell killing assay including an anti-CD2-amanitin ADC (i.e., RPA-2.10-AM or “CD2 RPA-2.10-AM”) having an interchain conjugated amanitin with drug-to-antibody ratio of 6. The results show the levels of viable NK-cells (y-axis) as a function of ADC (CD2 RPA-2.10-AM) or control antibody (i.e., hIgG1, hIgG1-amanitin (“hIgG1-AM”), or CD45-amanitin (“CD45 AM”)) concentration (x-axis), as assessed using a CellTiter Glo assay.

FIGS. 5A and 5B graphically depict the results of an in vivo T-cell depletion assay showing the absolute levels of T-cells (CD3+ cells; y-axis) in the peripheral blood (FIG. 5A) and bone marrow (FIG. 5B) of humanized NSG mice 7 days after a single administration of 0.3 mg/kg, 1 mg/kg, or 3 mg/kg of an anti-CD2-amanitin ADC (i.e., RPA-2.10-AM or “CD2 RPA-2.10-AM”) having an interchain drug-to-antibody ratio of 6. For comparison, FIGS. 5A and 5B also show the level of T-cell depletion following treatment of humanized NSG mice with 25 mg/kg of an unconjugated anti-CD2 antibody (i.e., “CD2 Abl”) or with the indicated controls (i.e., 25 mg/kg anti-CD52 antibody (clone YTH34.5); 3 mg/kg hIgG1-amanitin ADC (“hIgG1-AM”), 25 mg/kg hIgG1, or PBS).

FIGS. 6A-6C graphically depict the results of an in vivo T-cell depletion assay showing the absolute levels of T-cells (CD3+ cells; y-axis) in the peripheral blood (FIG. 6A), bone marrow (FIG. 6B), and thymus (FIG. 6C) of humanized NSG mice 7 days after a single administration of 1 mg/kg or 3 mg/kg of an anti-CD2-amanitin ADC (i.e., RPA-2.10-AM or “CD2 RPA-2.10-AM”) having a site-specific drug-to-antibody ratio of about 2. For comparison, FIGS. 6A-6C also show the level of T-cell depletion following treatment of humanized NSG mice with 3 mg/kg of an unconjugated anti-CD2 antibody (i.e., “CD2 RPA-2.10”) or with the indicated controls (i.e., 3 mg/kg hIgG1-amanitin-ADC (“hIgG1-AM”) or PBS).

### DETAILED DESCRIPTION

The present invention provides methods for promoting acceptance of an immune cell (either autologous or allogeneic) expressing a chimeric antigen receptor (CAR) in a human subject receiving CAR therapy by administering an anti-CD2 antibody drug conjugate (ADC) to the patient receiving the CAR therapy. The methods disclosed herein can be used to improve acceptance of autologous or allogeneic immune cells (e.g., T cells) without reliance on (or alternatively a reduced use of) lymphodepleting chemotherapy commonly used as a conditioning therapy to reduce rejection of the CAR expressing immune cells.

### I. Definitions

As used herein, the term “about” refers to a value that is within 5% above or below the value being described.

As used herein, the term “allogeneic”, when used in the context of transplantation, is used to define cells (or tissue or an organ) that are transplanted from a donor to a recipient of the same species, wherein the donor and recipient are not the same subject.

As used herein, the term “autologous” refers to cells or a graft where the donor and recipient are the same subject.

As used herein, the term “xenogeneic” refers to cells where the donor and recipient species are different.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a CAR and express the CAR on the cell surface. Isolated host cells and engineered cells may be used, for example, for enhancing an NK cell activity or a T lymphocyte activity, treatment of cancer, and treatment of autoimmune diseases. In an embodiment, the engineered cell includes immune cells, e.g., T-cells or Natural Killer (NK cells).

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), and antibody fragments so long as they exhibit the desired antigen-binding activity.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

An “intact” or “full length” antibody, as used herein, refers to an antibody having two heavy (H) chain polypeptides and two light (L) chain polypeptides interconnected by disulfide bonds.

As used herein, the term “anti-CD2 antibody” or “an antibody that binds to CD2” or an “anti-CD2 ADC” or “an ADC that binds to CD2” refers to an antibody or ADC that specifically binds to human CD2 as CD2 is found on the cell surface of cells, such as T cells. The amino acid sequence of human CD2 to which an anti-CD2 antibody (or anti-CD2 ADC) would bind is described below in SEQ ID NO: 7.

The term “specifically binds”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD2, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD2” or “specifically binds to CD2,” as used herein, refers to an antibody or that binds to CD2 and has a dissociation constant (K_{D}) of 1.0×10⁻⁷ M or less, as determined by surface plasmon resonance. In one embodiment, K_{D} is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of CD2.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and U.S. Pat. No. 4,816,397.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, the terms “chimeric antigen receptor” or “CAR” refer to a recombinant polypeptide comprising at least an extracellular domain capable of specifically binding an antigen, a transmembrane domain, and at least one intracellular signaling domain. Generally a CAR is a genetically engineered receptor that redirects cytotoxicity of immune effector cells toward cells presenting the given antigen. CARs are molecules that combine antibody-based specificity for a desired antigen (e.g., a tumor antigen) with a T cell receptor-activating intracellular domain to generate a chimeric protein that exhibits a specific cellular immune activity. In particular embodiments, CARs comprise an extracellular domain (also referred to as a binding domain or antigen-specific binding domain), a transmembrane domain, and an intracellular (cytoplasmic) signaling domain. Engagement of the antigen binding domain of the CAR with the target antigen on the surface of a target cell results in clustering of the CAR and delivers an activation stimulus to the CAR-containing cell. A main characteristic of a CAR is its ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors. In some embodiments, a CAR comprises an extracellular binding domain that specifically binds to a tumor antigen; a transmembrane domain; and one or more intracellular signaling domains. In various embodiments, a CAR comprises an extracellular binding domain that specifically binds human CD2; a transmembrane domain; and one or more intracellular signaling domains.

As used herein, the term “CAR therapy” refers to administration of an immune cell that has been engineered to express a CAR, to a human subject for the treatment of a given disease, e.g., cancer or an autoimmune disease. CAR therapy refers to the specific treatment of the patient with the engineered immune cells and is not intended to include therapies that commonly are used in conjunction with CAR cell treatment, e.g., lymphodepleting chemotherapy. Notably, where the term “cell” is used throughout, populations of cells are also included by the term unless otherwise specified. For example, as CAR therapy requires administration of a population of engineered cells.

As used herein, the term “combination” or “combination therapy” refers to the use of two (or more) therapies in a single human patient. The terms are not intended to refer to a combination composition. For example, described herein is a combination therapy comprising administering an anti-CD2 ADC and CAR therapy.

The term “conditioning” refers to the preparation of a patient in need of CAR therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy.

The term “deplete,” in the context of the effect of an anti-CD2 antibody or ADC on CD2-expressing cells, refers to a reduction in the number of or elimination of CD2-expressing cells.

As used herein, the term “effective amount” or “therapeutically effective amount” refers to an amount that is sufficient to achieve the desired result or to have an effect on an autoimmune disease or cancer.

As used herein, the terms “subject” and “patient” refer to an organism, such as a human, that receives treatment for a particular disease or condition as described herein.

As used herein “to treat” or “treatment”, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting acceptance of CAR expressing immune cells (allogeneic or autologous—both of which can cause immune reactions in a patient receiving CAR therapy). Insofar as the methods of the present invention are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present invention may occur prior to onset of a disease. The term does not imply that the disease state is completely avoided.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of CARs or include plasmids that contain regulatory sequences, such as promoter and enhancer regions, which direct gene transcription. Other useful vectors for antibody or CAR expression contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Notably, the term “conjugate” (when referring to a compound) is also referred to interchangeably herein as a “drug conjugate”, “antibody drug conjugate” or “ADC”. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, antigen-binding fragment thereof, or specific anti-CD2 antibody that binds CD2 known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, the term “microtubule-binding agent” refers to a compound which acts by disrupting the microtubular network that is essential for mitotic and interphase cellular function in a cell. Examples of microtubule-binding agents include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof.

As used herein, the term “amatoxin” refers to a member of the amatoxin family of peptides produced by Amanita phalloides mushrooms, or derivative thereof, such as a variant or derivative thereof capable of inhibiting RNA polymerase II activity. Amatoxins may be isolated from a variety of mushroom species (e.g., Amanita phalloides, Galerina marginata, Lepiota brunneo-incarnata) or may be prepared semi-synthetically or synthetically. A member of this family, α-amanitin, is described in Wieland, Int. J. Pept. Protein Res. 1983, 22(3):257-276. A derivative of an amatoxin may be obtained by chemical modification of a naturally occurring compound (“semi-synthetic”), or may be obtained from an entirely synthetic source. Synthetic routes to various amatoxin derivatives are disclosed in, for example, U.S. Pat. No. 9,676,702 and in Perrin et al., J. Am. Chem. Soc. 2018, 140, p. 6513-6517, each of which is incorporated by reference herein in their entirety with respect to synthetic methods for preparing and derivatizing amatoxins.

Amatoxins useful in conjunction with the compositions and methods described herein include amatoxins described in Formula (II), e.g., α-amanitin, β-amanitin, γ-amanitin, ε-amanitin, amanin, amaninamide, amanullin, amanullinic acid, or proamanullin. As described herein, amatoxins may be conjugated to an antibody, or antigen-binding fragment thereof, for instance, by way of a linker moiety (L) (thus forming an ADC). The structures of exemplary amatoxin-linker conjugates are represented by Formulas (III), (IIIA), and (IIIB). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp² double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or sp3 carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent.

Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

Wherever a substituent is depicted as a di-radical (i.e., has two points of attachment to the rest of the molecule), it is to be understood that the substituent can be attached in any directional configuration unless otherwise indicated.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the claims may comprise one or more asymmetric centers, and different diastereomers and/or enantiomers of each of the compounds may exist. The description of any compound in this description and in the claims is meant to include all enantiomers, diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the invention of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

The sections that follow provide a description of methods based on the administration of an anti-CD2 ADCs to a human patient to promote acceptance of an immune cell expressing a CAR in CAR therapy.

### II. Anti-CD2 and CAR Methods of Treatment

A challenge of chimeric antigen receptor (CAR) therapy is determining a means by which the engineered CAR expressing cells, e.g., CAR-T cells, can be accepted by a human recipient. Such acceptance of the engineered immune cells can impact the efficacy of the treatment and also results in adverse side effects to the patient.

Lymphodepleting chemotherapy is a traditional way of suppressing the recipient's immune system to improve acceptance, but commonly has adverse side effects. Described herein are methods of promoting acceptance of (CAR) expressing immune cells in human patients who are receiving CAR therapy. The methods described herein specifically target CD2+ cells, e.g., CD2+ T cells, in the human patient who is undergoing CAR therapy and ablates the CD2+ cells. The methods disclosed herein are more targeted than lymphodepleting chemotherapy and provide a means by which either autologous or allogeneic cells can be used.

Described herein are methods of administering anti-CD2 antibody-drug conjugates (ADCs) to deplete a population of CD2 specific immune cells within the patient receiving CAR therapy in order to facilitate the acceptance and efficacy of CAR-expressing immune cells. This selective depletion of specific CD2 expressing cells of the immune system improves overall and relapse-free patient survival while decreasing the risk of rejection of the CAR-expressing immune cell for treating autoimmune disorders or cancer.

The risk of rejection of a CAR expressing immune cell remains high following the administration of CAR cell therapies. The methods and compositions disclosed herein may be used to inhibit or prevent the rejection of a CAR cell in a human patient. The anti-CD2 ADCs may be used to selectively target activated T cells in a patient who will be receiving a CAR cell therapy. Anti-CD2 ADCs, as described herein, may also be used to reduce the risk of the rejection of a CAR cell by targeting and depleting CD2 positive cells in a human patient who has already received a CAR cell therapy.

The compositions and methods described herein may be used to deplete CD2+ cells, e.g., T cells, that are associated with CAR cell therapy rejection. The methods of the invention promote acceptance of an immune cell expressing a CAR in a human subject, e.g., a human subject having cancer or an autoimmune disease. In one embodiment, the method includes administering an anti-CD2 antibody drug conjugate (ADC) to a human subject who will be undergoing or has undergone CAR therapy, and administering a therapeutically effective amount of an immune cell expressing a CAR to the human subject.

The anti-CD2 ADC can be administered to the human patient in need thereof prior to, concomitantly with, or following administration of one or more CAR cell therapies. In one embodiment, an anti-CD2 ADC is administered to the human patient in need thereof prior to (e.g., about 3 days before, about 2 days before, about 12 hours before) administration of CAR cell therapies. A single dose of an anti-CD2 ADC may be administered to the human patient either prior to, after, or concomitantly with, administration of CAR cell therapies, where such single dose is sufficient to prevent or reduce the risk of depletion of the CAR expressing immune cell.

In one embodiment, an anti-CD2 ADC is administered to the human patient in need thereof about 3 days prior to administration of one or more CAR cell therapies. In one embodiment, an anti-CD2 ADC is administered to the human patient in need thereof about 2 days prior to administration of one or more CAR cell therapies. In one embodiment, an anti-CD2 ADC is administered to the human patient in need thereof about 1 day prior to administration of one or more CAR cell therapies. In one embodiment, an anti-CD2 ADC is administered to the human patient in need thereof about 20 hours prior to administration of one or more CAR cell therapies. In one embodiment, an anti-CD2 ADC is administered to the human patient in need thereof about 18 hours prior to administration of one or more CAR cell therapies. In one embodiment, an anti-CD2 ADC is administered to the human patient in need thereof 15 hours prior to administration of one or more CAR cell therapies. In one embodiment, an anti-CD2 ADC is administered to the human patient in need thereof about 12 hours prior to administration of one or more CAR cell therapies. In one embodiment, an anti-CD2 ADC is administered to the human patient in need thereof about 6 hours prior to administration of one or more CAR cell therapies. In one embodiment, an anti-CD2 ADC is administered to the human patient in need thereof about 4 hours prior to administration of one or more CAR cell therapies. In one embodiment, an anti-CD2 ADC is administered to the human patient in need thereof about 2 hours prior to administration of one or more CAR cell therapies.

In one embodiment, an anti-CD2 ADC is administered to the human patient in need thereof concomitantly with the administration of a CAR cell therapy.

In one embodiment, an anti-CD2 ADC is administered to the human patient in need thereof about 2 hours after administration of one or more CAR cell therapies. In one embodiment, an anti-CD2 ADC is administered to the human patient in need thereof about 4 hours after administration of one or more CAR cell therapies. In one embodiment, an anti-CD2 ADC is administered to the human patient in need thereof about 6 hours after administration of one or more CAR cell therapies. In one embodiment, an anti-CD2 ADC is administered to the human patient in need thereof about 12 hours after administration of one or more CAR cell therapies.

In some embodiments, the anti-CD2 ADC can be administered up to about 21 days prior to the administration of one or more CAR cell therapies, e.g., about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, about 1 day, about 24 hours, about 12 hours, about 6 hours, about 3 hours, about 2 hours, or about 1 hour prior to the administration of one or more CAR cell therapies.

In some embodiments, the anti-CD2 ADC can be administered about 12 hours after administration of CAR cell therapies, e.g., about 12 hours, about 11 hours, about 10 hours, about 9 hours, about 8 hours, about 7 hours, about 6 hours, about 5 hours, about 4 hours, about 3 hours, about 2 hours, about 1 hour after the administration of one or more CAR cell therapies. In some embodiments, the anti-CD2 ADC can be administered about 10 days after administration of CAR cell therapies, e.g., about 10 days, about 9 days, about 8 days, about 7 days, about 6 days, about 5 days, about 4 days, about 3 days, about 2 days, about 1 day after the administration of one or more CAR cell therapies.

In one embodiment, the anti-CD2 ADC is administered before the CAR expressing immune cells are administered to the human patient in need thereof. In one embodiment, the anti-CD2 ADC is administered to the human patient in combination with CAR therapy, where the anti-CD2 ADC is administered to the human subject about 12 hours to about 21 days before administration of the CAR expressing immune cells. In one embodiment, the anti-CD2 ADC is administered to the human patient in combination with CAR therapy, where the anti-CD2 ADC is administered to the human subject about 18 hours to about 20 days before administration of the CAR expressing immune cells. In one embodiment, the anti-CD2 ADC is administered to the human patient in combination with CAR therapy, where the anti-CD2 ADC is administered to the human subject about 20 hours to about 18 days before administration of the CAR expressing immune cells. In one embodiment, the anti-CD2 ADC is administered to the human patient in combination with CAR therapy, where the anti-CD2 ADC is administered to the human subject about 1 day to about 15 days before administration of the CAR expressing immune cells. In one embodiment, the anti-CD2 ADC is administered to the human patient in combination with CAR therapy, where the anti-CD2 ADC is administered to the human subject about 1 day to about 10 days before administration of the CAR expressing immune cells. In one embodiment, the anti-CD2 ADC is administered to the human patient in combination with CAR therapy, where the anti-CD2 ADC is administered to the human subject about 2 days to about 8 days before administration of the CAR expressing immune cells. In one embodiment, the anti-CD2 ADC is administered to the human patient in combination with CAR therapy, where the anti-CD2 ADC is administered to the human subject about 3 days to about 6 days before administration of the CAR expressing immune cells.

Overall levels of T cells in a biological sample from a human patient can be tested following administration of an anti-CD2 ADC, wherein a decrease in the overall number of T cells in a human patient following administration of the anti-CD2 ADC relative to the level prior to administration indicates efficacy of the anti-CD2 ADC for preventing rejection of the CAR cell therapy. In one embodiment, the level of endogenous T cells in a biological sample from the human patient is reduced by at least about 5%, at least about 10%, at least about 15%, at least about 20%, relative to the level of T cells in a biological sample (of the same type, e.g., blood) from the human patient just prior to administration of the anti-CD2 ADC. In one embodiment, the level of endogenous T cells in a biological sample from the human patient is reduced by about 5% to 25%, by about 5% to 20%, by about 5% to 15%, or by about 5% to 10%, relative to the level of T cells in a biological sample (of the same type, e.g., blood) from the human patient just prior to administration of the anti-CD2 ADC. In one embodiment, the level of endogenous T cells is determined one day or less prior to administration of the anti-CD2 ADC.

Levels of T cells can be determined according to standard methods known in the art, including, but not limited, to fluorescence-activated cell sorting (FACs) analysis or a hematology analyzer.

As described above, one of the advantages of the methods described herein is that lymphodepleting chemotherapeutic agents can be reduced in amount or not included in the conditioning regimen administered to a human patient having or planning on having CAR therapy. Lymphodepleting chemotherapeutic agents such as, but not limited to, fludarabine, cyclophosphamide, bendamustine, and/or pentostatin are commonly used as anti-rejection agents to promote CAR expressing cell acceptance in a human receiving CAR therapy. In certain embodiments, a human patient is administered an anti-CD2 ADC in combination with, e.g., prior to, administration of a CAR expressing immune cell (e.g., T cell) such that the human patient does not receive lymphodepleting chemotherapeutic agent, e.g., fludarabine and/or cyclophosphamide, prior to, concomitantly with, or following prior to, concomitantly with, or following administration of the CAR expressing immune cell.

The use of other immune depleting agents can also be avoided or reduced through the use of an anti-CD2 ADC as an agent to deplete a human subject's endogenous immune cells and reduce the risk of rejection of the CAR expressing immune cells. For example, alemtuzumab is commonly used as an anti-rejection agent in combination with CAR therapy to promote CAR expressing cell acceptance in the human receiving CAR therapy. In certain embodiments, a human patient is administered an anti-CD2 ADC in combination with, e.g., prior to, administration of a CAR expressing immune cell (e.g., T cell) such that the human patient does not receive alemtuzumab prior to, concomitantly with, or following administration of the CAR expressing immune cell.

In certain embodiments, an anti-CD2 ADC is used in combination with another therapy in order to promote tolerance of the CAR expressing immune cells. For example, an anti-CD2 ADC may also be administered to the human patient prior to the human patient receiving CAR therapy. The anti-CD2 ADC can be administered prior to, concomitantly with, or following the anti-CD2 ADC, where both the anti-CD2 ADC and the anti-CD2 ADC are administered to the human patient prior to CAR therapy.

The methods disclosed herein can be used both for autologous and allogeneic cells expressing CARs. Importantly, the anti-CD2 ADC conditioning methods described herein are useful for expanding the type of immune cell that can be used in CAR therapy by providing a means by which tolerance of an allogeneic cell can be provided. In one embodiment, the CAR expressing immune cell is an allogeneic cell or an autologous cell. Examples of the types of immune cells that may be engineered to express a CAR include, but are not limited to, an allogeneic T cell, an autologous T cell, an autologous NK cell, or an allogeneic NK cell.

In one embodiment, the anti-CD2 antibody-drug conjugate is used to deplete CD2 expressing donor cells, e.g., activated T cells expressing CD2, by administering the anti-CD2 antibody-drug conjugate after the administration of CAR cell therapies. In one embodiment, the CAR cell therapies comprise allogeneic cells.

The methods disclosed herein are particularly useful for the treatment of cancer or an autoimmune disease in a human subject having one of these disorders.

In one embodiment, the methods disclosed herein are used to treat cancer. More specifically, an anti-CD2 ADC is administered to a human subject having cancer in combination with CAR therapy. Examples of the types of cancer that can be treated using the methods disclosed herein include, but are not limited to, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

In one embodiment, the methods disclosed herein are used to treat an autoimmune disease. More specifically, an anti-CD2 ADC is administered to a human subject having an autoimmune disease in combination with CAR therapy. Examples of autoimmune diseases that can be treated using the combination methods disclosed herein include, but are not limited to, multiple sclerosis, Crohn's disease, ulcerative colitis, rheumatoid arthritis, type 1 diabetes, lupus, and psoriasis.

In certain embodiments, an anti-CD2 ADC is administered to a human patient in combination with a CAR-T cell therapy. In one embodiment, the anti-CD2 ADC is administered to the human patient prior to administration of the CAR-T therapy. Examples of CAR-T cells that could be used in combination with the anti-CD2 ADC therapy described herein include, but are not limited to, CD19 CAR-T (e.g., CART-19-01,02,03 (Fujian Medical University); daopeicart (Hebei Senlang Biotechnology Inc.); IM19CART/001, YMCART201702 (Beijing Immunochina Medical Science & Technology Co.); CART-CD19-02,03 (Wuhan Sian Medical Technology Co.); Universal CD19-CART/SHBYCL001,002 (Shanghai Bioray Laboratory Inc.); UnicarTherapy201701 (Shanghai Unicar-Therapy Biomedicine Technology Co.); Genechem/NCT02672501 (Shanghai GeneChem Co.); SenL_19 (Hebei Senlang Biotechnology Inc.); PCAR-019 (PersonGen BioTherapeutics (Suzhou); ICAR19 (Immune Cell, Inc.); WM-CART-02 (Sinobioway Cell Therapy Co.); HenanCH080,109,152 (Henan Cancer Hospital/The Pregene (ShenZhen) Biotechnology Co.); IM19-CD28 and IM19-41BB CAR-T cells (Beijing Immunochina Medical Science & Technology Company); CTL019/IT1601-CART19 (Beijing Sanwater Biological Technology Co.); CTL019/CCTL019C2201 (Novartis Pharmaceuticals); CD19:4-1BB:CD28:CD3/FirstShenzhen01 (Shenzhen Second People's Hospital/The Beijing Pregene Science and Technology Company); MB-CART19.1 (Shanghai Children's Medical Center/Miltenyi Biotec GmbH); PZ01 CAR-T cells (Pinze Lifetechnology Co.); YMCART201701 (Beijing Immunochina Medical Science & Technology Co.); 2016YJZ12 (Peking University/Marino Biotechnology Co.); EGFRt/19-28z/4-1BBL CAR T cells (Memorial Sloan Kettering Cancer Center/Juno Therapeutics, Inc.); Doing-002 (Beijing Doing Biomedical Co.); PCAR-019 (PersonGen BioTherapeutics (Suzhou) Co.); C-CAR011 (Peking Union Medical College Hospital/Cellular Biomedicine Group Ltd.); iPD1 CD19 eCAR T cells (Peking University/Marino Biotechnology Co.); 2013-1018/NCT02529813 (M. D. Anderson Cancer Center/Ziopharm/Intrexon Corp.); HenanCH CAR 2-1 (Henan Cancer Hospital/The Pregene (ShenZhen) Biotechnology Co.); JCAR015 (Juno Therapeutics, Inc.); JCAR017/017001,004,006 (Juno Therapeutics, Inc.); JCAR017 (Celgene); TBI-1501 (Takara Bio Inc.); JMU-CD19CAR (Jichi Medical University); KTE-C19 (Kite, A Gilead Company); TriCAR-T-CD19 (Timmune Biotech Inc.); PF-05175157 (Fred Hutchinson Cancer Research Center)); CD22/CD30/CD7/BCMA/CD123 (e.g., 2016040/NCT03121625 (Hebei Senlang Biotechnology Inc.)); CD22 (e.g., Ruijin-CAR-01 (Ruijin Hospital/Shanghai Unicar-Therapy Bio-medicine Technology Co.); AUTO-PA1, DB1 (Autolus Limited)), CD20 (e.g., Doing-006 (Beijing Doing Biomedical Co.)); or CD20/CD22/CD30 (e.g., SZ5601 (The First Affiliated Hospital of Soochow University Shanghai/Unicar-Therapy Bio-medicine Technology Co.)).

### Chimeric Antigen Receptors (CARs)

The present invention includes the use of CAR therapy in combination with an anti-CD2 immune suppressing ADC. The invention is not generally limited to a specific CAR construct, e.g., a specific antigen binding region or intracellular signaling domain, as the invention is based, at least in part, on the discovery that anti-CD2 ADCs can serve as a conditioning agent for CAR therapy by promoting acceptance of CAR expressing cells by ablating endogenous CD2+ immune cells, such as endogenous T cells. Specific CARs, e.g., CD19 specific CARs, are contemplated herein and are included in the methods disclosed herein, but are not meant to be limiting.

CAR constructs are known in the art and generally contain (a) an extracellular region comprising an antigen binding domain, (b) a transmembrane domain and (c) a cytoplasmic signaling domain. Exemplary CAR configurations are known in the art, and any suitable configuration can be used in the methods described herein. For example, the CAR may be a first generation, a second generation, or a third generation CAR, e.g., as described in Guedan et al. Molecular Therapy-Methods & Clinical Development. 12: 145-156 (2019) or Sadelain et al. Cancer discovery 3.4: 388-398 (2013), the entire contents of which are hereby incorporated by reference. Briefly, a “first generation” CAR can comprise an (a) extracellular antigen binding domain, (b) a transmembrane domain, (c) one or more intracellular signaling domains, and optionally (d) a hinge region connecting the antigen binding domain to the transmembrane domain. A “second generation” CAR can comprise elements (a), (b), (c), and optionally (d), and further includes a co-stimulatory domain, for example, a co-stimulatory domain of CD28 or 4-1BB. A “third generation” CAR can comprise elements (a), (b), (c), and optionally (d), and further includes multiple co-stimulatory domains, for example, the co-stimulatory domains of CD28 and 4-1BB, or the co-stimulatory domains of CD28 and OX40. Each of the foregoing elements is described in detail below. It should be appreciated that in some embodiments, CAR molecules described by the following exemplary, non-limiting arrangements are from left to right, N-terminus to C-terminus of the CAR. A CAR as described by the disclosure may comprise or further comprise any other combination of elements as described herein.

The CAR used in the methods disclosed herein can include an extracellular antigen binding domain. The extracellular antigen binding domain can be any molecule that binds to an antigen, including, but not limited to, a human antibody, a humanized antibody, or any a functional fragment thereof. In certain embodiments, the antigen binding domain is an scFv. In other embodiments, the extracellular antigen binding domain is a non-immunoglobulin scaffold protein. In other embodiments, the extracellular binding domain of the CAR comprises a single chain T cell receptor (scTCR). As described in U.S. Pat. Nos. 5,359,046, 5,686,281 and 6,103,521, the extracellular domain may also be obtained from any of the wide variety of extracellular domains or secreted proteins associated with ligand binding and/or signal transduction.

The choice of the molecular target (antigen) of the extracellular binding domain depends upon the type and number of ligands that define the surface of a target cell. For example, the antigen binding domain may be chosen to recognize a ligand that acts as a cell surface marker on target cells associated with a particular disease state. Thus, in one aspect, the CAR-mediated immune cell (e.g., T-cell) response can be directed to an antigen of interest by way of engineering an extracellular antigen binding domain that specifically binds a desired antigen into a CAR. For example, the antigen binding domain may be chosen to recognize a ligand that acts as a cell surface marker on target cells associated with a particular disease state, such as cancer or an autoimmune disease. Thus examples of cell surface markers that may act as ligands for the antigen binding domain in a CAR include those associated with cancer cells and other forms of diseased cells, for example, autoimmune disease cells and pathogen infected cells. In some embodiments, a CAR is engineered to target a tumor antigen of interest by way of engineering a desired antigen binding domain that specifically binds to an antigen on a tumor cell. In the context of the present invention, “tumor antigen” refers to antigens that are common to specific hyperproliferative disorders such as cancer. In one embodiment, the antigen is a tumor antigen, examples of which include, but are not limited to, CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123. In one embodiment, CAR comprises an scFv that binds to CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

In another aspect, the extracellular binding domain of the CAR binds to an antigen that is AFP (e.g., ETCH17AFPCAR01 (Aeon Therapeutics (Shanghai) Co./Eureka Therapeutics Inc.)), GPC3 (e.g., GeneChem GPC-3 CART (Shanghai GeneChem Co.); 302 GPC3-CART (Shanghai GeneChem Co.); CAR-T for liver cancer (Shanghai GeneChem Co.); CAR-GPC3 T cells (Carsgen Therapeutics)), MUC1 (e.g., PG-021-001,002 (PersonGen BioTherapeutics (Suzhou) Co.)), mesothelin (e.g., H2017-01-P01 (Ningbo Cancer Hospital); TAI-meso-CART (Shanghai GeneChem Co.); K16-4/NCT02930993 (China Meitan General Hospital/Marino Biotechnology Co.)), CD38 (e.g., Anti-CD38 A2 CAR-T/SOR-CART-MM-001 (Sorrento Therapeutics, Inc.)), herinCAR-PD1 (e.g., herinCAR-PD1/NBWYKY2016-06-001,002,003 (Ningbo Cancer Hospital); SIMC-20160101,02,03 (Shanghai International Medical Center)), BCMA (e.g., P-BCMA-101 autologous T stem cell memory (Tscm) CAR-T cells/P-BCMA-101-001 (Poseida Therapeutics, Inc.); HenanCH284 (Henan Cancer Hospital/The Pregene (ShenZhen) Biotechnology Company); LCAR-B38M CAR-T cells (Nanjing Legend Biotech Co.); 9762/NCT03338972 (Fred Hutchinson Cancer Research Center/Juno Therapeutics, Inc.); Descartes-08 (Cartesian Therapeutics); KITE-585 (Kite, A Gilead Company); bb21217 (bluebird bio); bb21217 (Celgene); JCARH125 (Juno Therapeutics, Inc.)), CD30 (e.g., ICAR30 T cells (Immune Cell, Inc.)), EGFR (e.g., EGFR:4-1BB:CD28:CD3 modified T cells/First Shenzhen02 (Shenzhen Sceond People's Hospital/The Beijing Pregene Science and Technology Company); EGFR-IL12-CART (Shenzhen Second People's Hospital/The Pregene (ShenZhen) Biotechnology Co.); SBNK-2016-015-01 (Beijing Sanbo Brain Hospital/Marino Biotechnology Co.)), MG7 (e.g., MG7-CART (Xijing Hospital/Shanghai GeneChem Co.)), BCMA/TACI (e.g., AUTO2-MM1 (Autolus Limited)), CEA (e.g., 383-74/NCT02416466 (Roger Williams Medical Center/Sirtex Medical)), mesothelin/PSCA/CEA/HER2/MUC1/EGFRvIII (e.g., NCT03267173 (First Affiliated Hospital of Harbin Medical University/Shanghai Unicar-Therapy Bio-medicine Technology Co.)), CD20 (e.g., EY201605-19 (Beijing Biohealthcare Biotechnology Co.)), CD33 (e.g., 2016-0341/NCT03126864 (M. D. Anderson Cancer Center/Intrexon Corp./Ziopharm)), EGFR/BCMA (e.g., EGFRt/BCMA-41BBz CAR T cell (Memorial Sloan Kettering Cancer Center/Juno Therapeutics, Inc.)), ROR2 (e.g., autologous CCT301-38 or CCT301-59 T cells (Shanghai Sinobioway Sunterra Biotech)), NKR-2 (e.g., CYAD-N2T-002,003,004 (Celyad)), PSCA (e.g., BP-012 (Bellicum Pharmaceuticals)), CD28 (e.g., autologous CSR T cells (Beijing Sanbo Brain Hospital/Marino Biotechnology Co.)), TAA (e.g., AMG 119 (Amgen)), NKG2D (e.g., CM-CS1 (Celyad)), or CD123 (e.g., UCART123 (Cellectis S.A.)). The foregoing sentence further provides examples of CARs that bind said antigens (e.g., AMG 119 (Amgen)). These CAR constructs may be used in the conditioning methods disclosed herein with an anti-CD2 ADC.

A CAR construct can further contain a transmembrane domain that connects (either literally or by general proximity, e.g., with spacers) the extracellular antigen binding domain to a cytoplasmic signaling domain. In some embodiments, the extracellular antigen binding domain (e.g., a scFv, Fab or other antigen binding moiety) of a CAR can be linked to a transmembrane domain using a hinge or other linker. A spacer, linker, or hinge can be introduced between the extracellular antigen binding domain and the transmembrane domain to provide the flexibility to allow the antigen-binding domain to orient in different directions, thereby facilitating antigen recognition and binding. The cytoplasmic side of the transmembrane domain can be attached to an intracellular signaling domain, such as the intracellular signaling domain of CD28 or CD3 zeta (CD3-ζ), and can additionally include one or more co-stimulatory domains as discussed below.

Thus, in certain embodiments, the CAR can further comprise a hinge region positioned between the extracellular antigen binding domain and the transmembrane domain. For example, the hinge region can be derived from the hinge region of IgG1, IgG2, IgG3, IgG4, IgA, IgD, IgE, IgM, CD28, or CD8 alpha. In one particular embodiment, the hinge region is derived from the hinge region of IgG4. In another embodiment, the hinge region is a CD8 hinge domain (see SwissProt/GenBank Acc. No. P01732).

In one embodiment, a CAR comprises an extracellular antigen binding domain and a transmembrane domain connected via a CD8 hinge: AKPTTTPAPR PPTPAPTIAS QPLSLRPEAC RPAAGGAVHT RGLDFA (SEQ ID NO 8).

In one embodiment, a CAR comprises an extracellular antigen binding domain and a transmembrane domain connected via a hybrid CD8-CD28 hinge: AKPTTTPAPR PPTPAPTIAS QPLSLRPEAC RPAAGGAVHT RGLDFAPRKI EVMYPPPYLD NEKSNGTIIH VKGKHLCPSP LFPGPSKP (SEQ ID NO: 9).

The transmembrane domain may be derived from the sequence of a protein contributing the extracellular antigen binding domain, a protein contributing the effector function signaling domain, a protein contributing the proliferation signaling portion, or by a totally different protein. In some embodiments, the transmembrane domain is naturally associated with one of the other domains of the CAR. For example, the transmembrane domain and the cytoplasmic domain can be derived from the transmembrane region and the cytoplasmic region of the same protein. In one embodiment, the transmembrane and cytoplasmic domains of the CAR comprise contiguous portions of the CD28 sequence. Any transmembrane domain may be used in the CAR constructs described herein, provided that the domain is capable of anchoring a CAR comprising the antigen binding domain to a cell membrane.

The transmembrane domain may be derived either from a natural or from a synthetic source. Where the source is natural, the domain may be derived from any membrane-bound or transmembrane protein. Exemplary transmembrane domains that can be used in the methods provided herein may be derived from (e.g., comprise at least the transmembrane domain(s) of) the alpha, beta or zeta chain of the T-cell receptor, CD28, CD3 epsilon, CD45, CD4, CD2, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and fragments thereof. The transmembrane domain of a protein can be identified using any method known in the art, e.g., hydrophobicity analysis, structural analysis, etc., or by using public databased, e.g., the UniProt Database.

In some embodiments, the transmembrane domain may be synthetic. In exemplary embodiments, the transmembrane domain can comprise predominantly hydrophobic residues such as leucine and valine. In one embodiment, a triplet of phenylalanine, tryptophan and valine can be positioned at each end of a synthetic transmembrane domain. Optionally, a short oligo- or polypeptide linker, preferably between 2 and 10 amino acids in length may form the linkage between the transmembrane domain and the cytoplasmic signaling domain of a CAR. A glycine-serine doublet provides a particularly suitable linker.

In some embodiments, the transmembrane domain in the CAR of the invention is the CD8 transmembrane domain. Sequences of CD8 for this purposes are taught in PCT Publication No. WO2014/055771A1.

In some embodiments, the transmembrane domain in the CAR is the CD8 transmembrane domain, or a functional portion thereof. For example, a CAR can comprise a CD3 transmembrane domain having an amino acid sequence of LDPKLCYLLD GILFIYGVIL TALFLRVK (SEQ ID NO: 10), or a functional portion thereof, such as LCYLLDGILF IYGVILTALF L (SEQ ID NO: 11).

In some embodiments, the transmembrane domain in the CAR of the invention is a CD28 transmembrane domain. An exemplary sequence of CD28 is provided below, as well as an exemplary transmembrane domain sequence. In some embodiments, the CD28 transmembrane domain comprises the exemplary transmembrane domain sequence below, or a fragment or variant thereof that is capable of anchoring a CAR comprising the sequence to a cell membrane.

Thus, in some embodiments, the transmembrane domain of the CAR is a CD28 transmembrane domain containing the following amino acid sequence: FWVLVVVGGVLACYSLLVTVAFIIFWV (SEQ ID NO: 12). In one embodiment, the transmembrane domain of the CAR is a CD28 transmembrane domain containing the following amino acid sequence: IEVMYPPPYL DNEKSNGTII HVKGKHLCPS PLFPGPSKPF WVLVVVGGVL ACYSLLVTVA FIIFWV (SEQ ID NO: 13), or a functional fragment thereof, e.g., SEQ ID NO: 12.

In addition to an extracellular antigen binding domain and a transmembrane domain, a CAR further comprises an intracellular (or cytoplasmic) signaling domain.

It is known that signals generated through the endogenous TCR alone are insufficient for full activation of the T cell and that a secondary or co-stimulatory signal may also be required. Thus, T cell activation can be mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences).

An “intracellular signaling domain” or “cytoplasmic signaling domain” as the terms are used herein, refers to an intracellular portion of a molecule. The intracellular signaling domain can generate a signal that promotes an immune effector function of the CAR containing immune cell, e.g., a CAR-T cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In one embodiment, the intracellular signaling domain of the CAR contains a CD3 zeta signaling region as described in SEQ ID NO: 14, or a signaling portion thereof.

(SEQ ID NO: 14)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

Cytoplasmic signaling domains further can include, but are not limited to, those derived from CD3 zeta, FcR gamma, FcR beta, CD3 gamma, CD3 delta, CD3 epsilon, CDS, CD22, CD79a, CD79b, CD278 (“ICOS”), Fc.epsilon.RI, CD66d, DAP10, and DAP12.

A CAR may further contain an “intracellular costimulatory domain” which is a polypeptide chain derived from an intracellular signaling domain of a costimulatory protein or proteins, such as CD28 and 4-1BB, that enhance cytokine production.

Exemplary co-stimulatory signaling regions include 4-1BB, CD21, CD28, CD27, CD127, ICOS, IL-15Rα, and OX40.

In certain embodiments, the cytoplasmic costimulatory domain of a CAR comprises the 4-1BB signaling domain by itself or combined with any other desired cytoplasmic domain(s) useful in the context of a CAR. 4-1BB is a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank acc no. AAA62478.2, or the equivalent residues from a non-human species, e.g., mouse, rodent, monkey, ape and the like.

In one embodiment, the intracellular costimulatory signaling domain of the CAR is a 4-1BB (CD137) co-stimulatory signaling region, or a signaling portion thereof:

(SEQ ID NO: 15)
KRGRKKLLYIFKQPFMRPVQTTQEEDGCSCRFPEEEEGGCEL

In one embodiment, the costimulatory signaling domain of the CAR is a CD28 co-stimulatory signaling region sequence. For example, the costimulatory signaling domain can comprise the following CD28 co-stimulatory signaling region, or a signaling portion thereof:

(SEQ ID NO: 16)
RSKRSRLLHSDYMNMTPRRPGPTRKHYQPYAPPRDFAAYRS.

In exemplary embodiments, the cytoplasmic domain of the CAR can contain a CD3-zeta signaling domain, in combination with any other desired cytoplasmic domain(s) useful in the context of the CAR of the invention. In certain embodiments, the cytoplasmic domain of the CAR can comprise a CD3 zeta domain and a costimulatory signaling region, including, but not limited to, a costimulatory signaling region of 4-1BB, CD28, and CD27.

The cytoplasmic signaling sequences within the cytoplasmic signaling portion of the CAR of the invention may be linked to each other in a random or specified order. Optionally, a short oligo- or polypeptide linker or spacer, preferably between 5 and 20 amino acids in length may be inserted between cytoplasmic domains. A GGGGS (SEQ ID NO: 17) or (GGGGS)×3 (SEQ ID NO: 18) provides a particularly suitable linker.

In one embodiment, a CAR used herein includes an extracellular domain containing a single chain variable domain of an anti-CD19 monoclonal antibody, a transmembrane domain containing a hinge and transmembrane domain of CD8α, and a cytoplasmic domain containing the signaling domain of CD3ζ and the signaling domain of 4-1BB. An exemplary CAR includes an extracellular domain include the anti-CD19 monoclonal antibody which is described in Nicholson I C, et al., Mol Immunol 34:1157-1165 (1997) plus the 21 amino acid signal peptide of CD8α (translated from 63 nucleotides at positions 26-88 of GenBank Accession No. NM_001768). The CD8α hinge and transmembrane domain consists of 69 amino acids translated from the 207 nucleotides at positions 815-1021 of GenBank Accession No. NM_001768. The CD3ζ signaling domain of the preferred embodiment contains 112 amino acids translated from 339 nucleotides at positions 1022-1360 of GenBank Accession No. NM_000734.

Between the extracellular domain (comprising the antigen binding domain) and the transmembrane domain of the CAR, or between the cytoplasmic domain and the transmembrane domain of the CAR, there may be incorporated a spacer or hinge domain. As used herein, the term “spacer domain” generally means any oligo- or polypeptide that functions to link the transmembrane domain to the extracellular domain and/or the cytoplasmic domain in the polypeptide chain. As used herein, a hinge domain generally means any oligo- or polypeptide that functions to provide flexibility to the CAR, or domains thereof, and/or prevent steric hindrance of the CAR, or domains thereof. In some embodiments, a spacer or hinge domain may comprise up to 300 amino acids, preferably 10 to 100 amino acids and most preferably 5 to 20 amino acids. It also should be appreciated that one or more spacer domains may be included in other regions of a CAR, as aspects of the disclosure are not limited in this respect.

It is to be understood that a CAR can include a region (e.g., an antigen binding domain, a transmembrane domain, a cytoplasmic domain, a signaling domain, a safety domain, and/or a linker, or any combination thereof) having a sequence provided herein or a variant thereof or a fragment of either one thereof (e.g., a variant and/or fragment that retains the function required for the CAR activity) can be included in a CAR protein as described herein. In some embodiments, a variant has 1, 2, 3, 4, 5, 6, 7, 8, 9, or 10 amino acid changes relative to the illustrated sequence. In some embodiments, a variant has a sequence that is at least 80%, at least 85%, at least 90%, 90%-95%, at least 95% or at least 99% identical to the illustrated sequence. In some embodiments, a fragment is 1-5, 5-10, 10-20, 20-30, 30-40, or 40-50 amino acids shorter than a sequence provided herein. In some embodiments, a fragment is shorter at the N-terminal, C-terminal, or both terminal regions of the sequence provided. In some embodiments, a fragment contains 80%-85%, 85%-90%, 90%-95%, or 95%-99% of the number of amino acids in a sequence provided herein.

In other embodiments, the invention comprises nucleic acid sequences that encode for the amino acid sequences disclosed herein.

In some embodiments, the above exemplary, non-limiting arrangements are from left to right, N-terminus to C-terminus of the CAR. The CAR may comprise or further comprise any other combination of elements as described herein.

Once the CAR construct is identified with its various parts, a CAR expressing immune cell is produced whereby the immune cell expresses the CAR. The method includes introducing into, e.g., transducing, the immune cell with a nucleic acid molecule described herein (e.g., an RNA molecule, e.g., an mRNA), or a vector comprising a nucleic acid molecule encoding a CAR, e.g., a CAR described herein. The present invention also provides a method of generating a population of cells (e.g., RNA-engineered cells transiently expressing an exogenous RNA). The method includes introducing into the cell an RNA as described herein (e.g., an in vitro transcribed RNA or synthetic RNA; an mRNA sequence encoding a CAR polypeptide as described herein). In embodiments, the RNA expresses the CAR polypeptide transiently. In one embodiment, the cell is a cell as described herein, e.g., an immune effector cell (e.g., T cells or NK cells, or cell population).

Other exemplary chimeric antigen receptor constructs are disclosed in U.S. Pat. Nos. 9,328,156; 9,783,591; 9,714,278; 9,765,156; 10,117,896; 9,573,988; 10,308,717; 10,221,245; 10,040,865; U.S. Patent Publication No. 2018/0256712A1; U.S. Patent Publication No. 2018/0271907A1; U.S. Patent Publication No. 2016/0046724A1; U.S. Patent Publication No. 2018/0044424A1; U.S. Patent Publication No. 2018/0258149A1; U.S. Patent Publication No. 2019/0151363A1; and U.S. Patent Publication No. 2018/0273601A1; the contents of each of the foregoing patents and patent publications are incorporated by reference herein in their entirety.

### III. Anti-CD2 Antibody Drug Conjugates (ADCs)

As described herein, anti-CD2 ADCs can be used in combination with CAR therapy to treat cancer or an autoimmune disease in a human patient. More specifically, anti-CD2 ADCs can be used to deplete CD2+ cells (e.g., CD2+ T cells) in a human subject who is also receiving CAR therapy. Anti-CD2 ADCs target endogenous T cells and kill these cells such that the patient's immune system will not attack the CAR expressing immune cells (autologous or allogeneic) administered to the subject. Thus, anti-CD2 ADCs are used as a conditioning step in combination with CAR therapy to promote acceptance of the engineered CAR expressing immune cells in the recipient patient. One advantage of using anti-CD2 ADCs as a conditioning regimen is that endogenous T cells expressing CD2 can be specifically targeted for depletion versus more traditional methods of conditioning for CAR therapy where general lymphodepleting chemotherapeutic agents are administered to the subject.

Examples of anti-CD2 ADCs and conditioning methods using the same are described in PCT publication WO 2019/108860, incorporated by reference herein.

### Anti-CD2 Antibodies

ADCs capable of binding CD2 can be used as therapeutic agents to promote acceptance in a human patient of immune cells expressing CARs by preventing or reducing the risk of rejection of the immune cells expressing CARs.

The anti-CD2 ADCs described herein include an anti-CD2 antibody or antigen binding portion thereof, linked to a cytotoxin.

Human CD2 is also referred to as T-cell Surface Antigen T11/Leu-5, T11, CD2 antigen (p50), and Sheep Red Blood Cell Receptor (SRBC). CD2 is expressed on T cells. Two isoforms of human CD2 have been identified. Isoform 1 contains 351 amino acids is described in Seed, B. et al. (1987) 84: 3365-69 (see also Sewell et al. (1986) 83: 8718-22) and below (NCBI Reference Sequence: NP_001758.2):

(SEQ ID NO: 7)
msfpckfvas fllifnvssk gavskeitna letwgalgqd
inldipsfqm sddiddikwe ktsdkkkiaq frkeketfke
kdtyklfkng tlkikhlktd dqdiykvsiy dtkgknvlek
ifdlkiqerv skpkiswtci nttltcevmn gtdpelnlyq
dgkhlklsqr vithkwttsl sakfkctagn kvskessvep
vscpekgldi yliigicggg sllmvfvall vfyitkrkkq
rsrrndeele trahrvatee rgrkphqipa stpqnpatsq
hpppppghrs qapshrpppp ghrvqhqpqk rppapsgtqv
hqqkgpplpr prvqpkpphg aaenslspss n

A second isoform of CD2 is 377 amino acids and is identified herein as NCBI Reference Sequence: NP_001315538.1.

In one embodiment, an anti-CD2 antibody that may be used in conjunction with the compositions and methods described herein include those that have one or more, or all, of the following CDRs:

a. a CDR-H1 having the amino acid sequence
(SEQ ID NO: 1)
EYYMY;
b. a CDR-H2 having the amino acid sequence
(SEQ ID NO: 2)
RIDPEDGSIDYVEKFKK;
c. a CDR-H3 having the amino acid sequence
(SEQ ID NO: 3)
GKFNYRFAY;
d. a CDR-L1 having the amino acid sequence
(SEQ ID NO: 4)
RSSQSLLHSSGNTYLN;
e. a CDR-L2 having the amino acid sequence
(SEQ ID NO: 5)
LVSKLES;
and
f. a CDR-L3 having the amino acid sequence
(SEQ ID NO: 6)
MQFTHYPYT.

Antibodies and antigen-binding fragments thereof containing the foregoing CDR sequences are described, e.g., in U.S. Pat. No. 6,849,258, the disclosure of which is incorporated herein by reference as it pertains to anti-CD2 antibodies and antigen-binding fragments thereof.

Further, in certain embodiments the anti-CD2 ADC has a serum half-life in a human subject of 3 days or less.

Additional anti-CD2 antibodies that can be used in the ADCs described herein can be identified using techniques known in the art, such as hybridoma production. Hybridomas can be prepared using a murine system. Protocols for immunization and subsequent isolation of splenocytes for fusion are known in the art. Fusion partners and procedures for hybridoma generation are also known. Alternatively, anti-CD2 antibodies can be generated using the HuMAb-Mouse® or XenoMouse™. In making additional anti-CD2 antibodies, the CD2 antigen is isolated and/or purified. The CD2 antigen may be a fragment of CD2 from the extracellular domain of CD2. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD2 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD2 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD2 antibodies for use in the anti-CD2 ADCs described herein can also be identified using high throughput screening of libraries of antibodies or antibody fragments for molecules capable of binding CD2. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules).

In addition to in vitro display techniques, computational modeling techniques can be used to design and identify anti-CD2 antibodies or antibody fragments in silico, for instance, using the procedures described in US 2013/0288373, the disclosure of which is incorporated herein as it pertains to molecular modeling methods for identifying anti-CD2 antibodies. For example, using computational modeling techniques, one of skill in the art can screen libraries of antibodies or antibody fragments in silico for molecules capable of binding specific epitopes on CD2, such as extracellular epitopes of CD2.

In one embodiment, the anti-CD2 antibody used in the ADCs described herein are able to internalize into the cell. In identifying an anti-CD2 antibody (or fragment thereof) additional techniques can be used to identify antibodies or antigen-binding fragments that bind CD2 on the surface of a cell (e.g., a T cell) and further are able to be internalized by the cell, for instance, by receptor-mediated endocytosis. For example, the in vitro display techniques described above can be adapted to screen for antibodies or antigen-binding fragments thereof that bind CD2 on the surface of a hematopoietic stem cell and that are subsequently internalized. Phage display represents one such technique that can be used in conjunction with this screening paradigm. To identify anti-CD2 antibodies or fragments thereof that bind CD2 and are subsequently internalized a CD2+ cell, one of skill in the art can use the phage display techniques described in Williams et al., Leukemia 19:1432-1438, 2005, the disclosure of which is incorporated herein by reference in its entirety.

The internalizing capacity of an anti-CD2 antibody or fragment thereof can be assessed, for instance, using radionuclide internalization assays known in the art. For example, an anti-CD2 antibody or fragment thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with hematopoietic stem cells for a time sufficient to permit internalization. Internalized antibodies, or fragments thereof, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

In some embodiments, the anti-CD2 antibody (or fragment thereof) has a defined serum half-life. For example, an anti-CD2 antibody (or fragment thereof) may have a serum half-life of about 1-24 hours in the human patient. ADCs containing such anti-CD2 antibodies can also, for example, have a serum half-life of about 1-24 hours in a human patient. Pharmacokinetic analysis by measurement of serum levels can be performed by assays known in the art.

For recombinant production of an anti-CD2 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Cytotoxins

Various cytotoxins can be conjugated to an anti-CD2 antibody via a linker for use in the combination therapies described herein. In particular, the anti-CD2 ADCs include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

ADCs of the present invention therefore may be of the general Formula I, wherein an antibody or antigen-binding fragment thereof (Ab) is conjugated (covalently linked) to linker (L), through a chemical moiety (Z), to a cytotoxic moiety (“drug,” D).

Ab-(Z-L-D)ₙ (I)

Accordingly, the antibody or antigen-binding fragment thereof may be conjugated to a number of drug moieties as indicated by integer n, which represents the average number of cytotoxins per antibody, which may range, e.g., from about 1 to about 20. Any number of cytotoxins can be conjugated to the antibody, e.g., about 1, about 2, about 3, about 4, about 5, about 6, about 7, or about 8. In some embodiments, n is from 1 to 4. In some embodiments, n is from 1 to 3. In some embodiments, n is about 2. In some embodiments, n is about 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-CD2 ADCs, n may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof, or another cytotoxic compound described herein or known in the art.

In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof.

Additional details regarding cytotoxins that can be used in the anti-CD2 ADCs useful in the methods of the invention are described below.

Amatoxins

In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof. Structures of the various naturally occurring amatoxins are represented by Formula II and accompanying Table 1, and are disclosed in, e.g., Zanotti et al., Int. J. Peptide Protein Res. 30, 1987, 450-459.

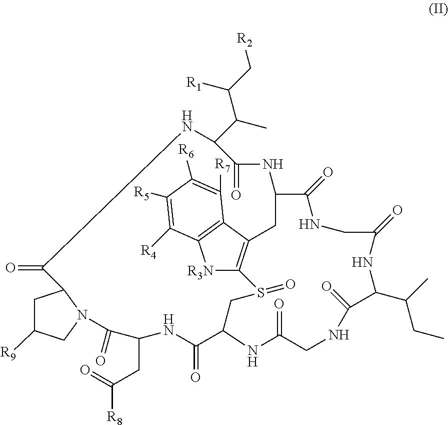

TABLE 1
Naturally occurring Amatoxin structure table.

| Name | R₁ | R₂ | R₃, R₄ | R₅ | R₆, R₇ | R₈ | R₉ |
|---|---|---|---|---|---|---|---|
| α-amanitin | OH | OH | H | OH | H | NH₃ | OH |
| β-amanitm | OH | OH | H | OH | H | OH | OH |
| γ-amamtin | OH | H | H | OH | H | NH₃ | OH |
| ε-amanitm | OH | H | H | OH | H | OH | OH |
| Amanin | OH | OH | H | H | H | OH | OH |
| Amaninamide | OH | OH | H | H | H | NH₃ | OH |
| Amanullin | H | H | H | OH | H | NH₃ | OH |
| Amanullinic | H | H | H | OH | H | OH | OH |
| acid | | | | | | | |
| Proamanullin | H | H | H | OH | H | NH₃ | H |

In one embodiment, the cytotoxin is an amanitin or derivative thereof. In one embodiment, the cytotoxin is an α-amanitin or derivative thereof.

Many positions on amatoxins or derivatives thereof can serve as the position to covalently bond the linking moiety L, and, hence the antibodies or antigen-binding fragments thereof. In some embodiments, the cytotoxin in the ADC of Formula I is an amatoxin or derivative thereof according to formula (II), In one embodiment, the ADC is represented by the formula Ab-Z-L-Am, wherein Ab is an antibody or antigen-binding fragment thereof that binds CD2, L is a linker, Z is a chemical moiety, and Am is an amatoxin. In this embodiment, the linker-amatoxin conjugate Am-L-Z is represented by Formula (III)

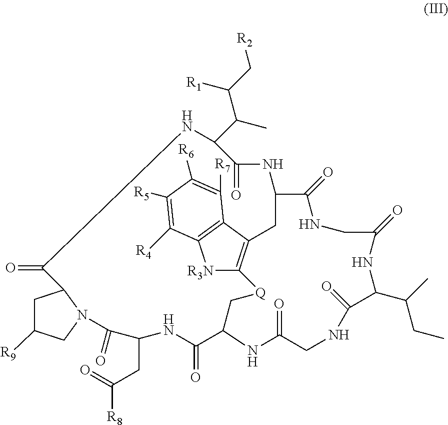

wherein:

R₁ is H, OH, OR_{A}, or OR_{C};

R₂ is H, OH, OR_{B}, or OR_{C};

R_{A} and R_{B}, when present, together with the oxygen atoms to which they are bound, combine to form a 5-membered heterocycloalkyl group;

R₃ is H, R_{C}, or R_{D};

each of R₄, R₅, R₆, and R₇ is independently H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

Q is —S—, —S(O)—, or —SO₂—;

R_{C} is -L-Z′ or -L-Z-Ab, wherein L is a linker, and is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl; comprises a dipeptide; or —((CH₂)ₘO)ₙ(CH₂)ₘ—, where m and n are each independently selected from 1, 2, 3, 4, 5, 6, 7, 8, 9, and 10; Z′ is a reactive moiety; and Z is a chemical moiety resulting from a coupling reaction of Z′ with a functional group on Ab; and

R_{D} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination thereof, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro. In some embodiments, the amatoxin contains one R_{C} substituent.

Formula (III) includes an amatoxin and a linker and, in some embodiments, a linker, a chemical moiety, and an antibody.

In some embodiments, the cytotoxin is an amatoxin, and the linker-amatoxin conjugate or the antibody-linker-amatoxin conjugate is represented by formula (IIIA):

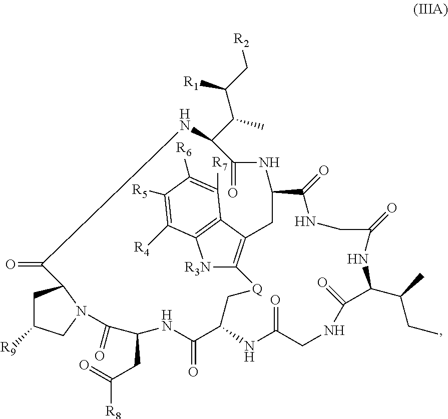

wherein:

R₁ is H, OH, OR_{A}, or OR_{C};

R₂ is H, OH, OR_{B}, or OR_{C};

R_{A} and R_{B}, when present, together with the oxygen atoms to which they are bound, combine to form a 5-membered heterocycloalkyl group;

R₃ is H, R_{C}, or R_{D};

each of R₄, R₅, R₆, and R₇ is independently H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

Q is —S—, —S(O)—, or —SO₂—;

R_{C} is -L-Z′ or -L-Z-Ab, wherein L is a linker, and is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl; or comprises a dipeptide; or —((CH₂)ₘO)ₙ(CH₂)ₘ, where m and n are each independently selected from 1, 2, 3, 4, 5, 6, 7, 8, 9, and 10; Z′ is a reactive moiety, and Z is a chemical moiety resulting from a coupling reaction of Z′ with a functional group on Ab; and

R_{D} is C₁-C₆ alkyl, C₁-C₆ heteroalkyl, C₂-C₆ alkenyl, C₂-C₆ heteroalkenyl, C₂-C₆ alkynyl, C₂-C₆heteroalkynyl, cycloalkyl, heterocycloalkyl, aryl, heteroaryl, or a combination thereof, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro. In some embodiments, the amatoxin contains one R_{C} substituent.

In some embodiments, R_{A} and R_{B}, together with the oxygen atoms to which they are bound, combine to form a 5-membered heterocycloalkyl group of formula:

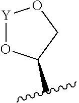

wherein Y is —(C═O)—, —(C═S)—, —(C═NR_{E})—, or —(CR_{E}R_{E′})—; and

wherein R_{E} and R_{E′} are each independently H, C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C}, or a combination thereof; wherein each C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C} is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

Formula (IIIA) includes an amatoxin and a linker and, in some embodiments, a linker, a chemical moiety, and an antibody.

In some embodiments, the cytotoxin is an amatoxin or derivative thereof and the amatoxin-linker conjugate is represented by formula IIIA, wherein

R₁ is H, OH, OR_{A}, or OR_{C};

R₂ is H, OH, OR_{B}, or OR_{C};

R_{A} and R_{B}, when present, together with the oxygen atoms to which they are bound, combine to form:
wherein R₃ is H or R_{C}.

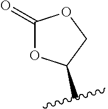 [SMILES: *[C@@H]1COC(=O)O1]

In some embodiments, the cytotoxin is an amatoxin or derivative thereof and the amatoxin-linker conjugate is represented by formula IIIA, wherein

R₁ is H, OH, OR_{A}, or OR_{C};

R₂ is H, OH, OR_{B}, or OR_{C};

R_{A} and R_{B}, when present, together with the oxygen atoms to which they are bound, combine to form:

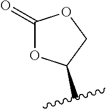

wherein

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH; and

wherein R_{C} and R_{D} are as defined above.

In some embodiments, the cytotoxin is an amatoxin or derivative thereof and the amatoxin-linker conjugate is represented by formula IIIA, wherein:

R₁ is H, OH, or OR_{A};

R₂ is H, OH, or OR_{B};

R_{A} and R_{B}, when present, together with the oxygen atoms to which they are bound, combine to form:

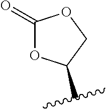

wherein

R₃, R₄, R₆, and R₇ are each H;

R₅ is OR_{C};

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2016/0002298, the disclosure of which is incorporated herein by reference in its entirety.

In some embodiments, the cytotoxin is an amatoxin or derivative thereof and the amatoxin-linker conjugate is represented by formula IIIA, wherein:

R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2014/0294865, the disclosure of which is incorporated herein by reference in its entirety.

In some embodiments, the cytotoxin is an amatoxin or derivative thereof and the amatoxin-linker conjugate is represented by formula IIIA, wherein:

R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2015/0218220, the disclosure of which is incorporated herein by reference in its entirety.

In some embodiments, the cytotoxin is an amatoxin or derivative thereof represented by formula IIIA, wherein:

R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH;

Q is —S—, —S(O)—, or —SO₂—; and

wherein R_{C} and R_{D} are as defined above. Such amatoxin conjugates are described, for example, in U.S. Pat. Nos. 9,233,173 and 9,399,681, the disclosures of each of which are incorporated herein by reference in their entirety.

In some embodiments, the cytotoxin is an amatoxin or derivative thereof and the amatoxin-linker conjugate is represented by formula IIIB:

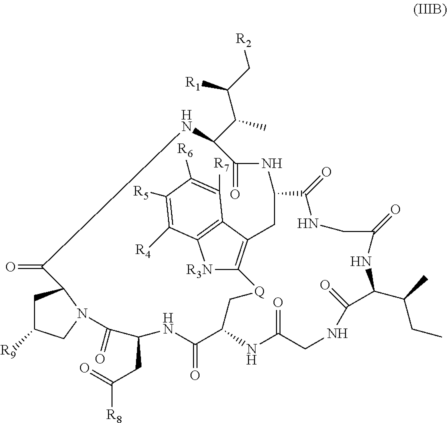

wherein:

R₁ is H, OH, OR_{A}, or OR_{C};

R₂ is H, OH, OR_{B}, or OR_{C};

R_{A} and R_{B}, when present, together with the oxygen atoms to which they are bound, combine to form a 5-membered heterocycloalkyl group;

R₃ is H, R_{C}, or R_{D};

each of R₄, R₅, R₆, and R₇ is independently H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

Q is —S—, —S(O)—, or —SO₂—;

R_{C} is -L-Z′ or -L-Z-Ab, wherein L is a linker, and is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl; or comprises a dipeptide; or —((CH₂)ₘO)ₙ(CH₂)ₘ, where m and n are each independently selected from 1, 2, 3, 4, 5, 6, 7, 8, 9, and 10; Z′ is a reactive moiety, and Z is a chemical moiety resulting from a coupling reaction of Z′ with a functional group on Ab; and

R_{D} is C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, heteroaryl, or a combination thereof, wherein each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, cycloalkyl, heterocycloalkyl, aryl, or heteroaryl is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

Formula (IIIB) includes an amatoxin and a linker and, in some embodiments, a linker, a chemical moiety, and an antibody.

In some embodiments, R_{A} and R_{B}, together with the oxygen atoms to which they are bound, combine to form a 5-membered heterocycloalkyl of formula:

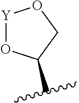

wherein Y is —(C═O)—, —(C═S)—, —(C═NR_{E})—, or —(CR_{E}R_{E′})—; and

wherein R_{E} and R_{E′} are each independently H, C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C}, or a combination thereof, wherein each C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C} is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, the antibodies or antigen-binding fragments thereof as described herein are conjugated to an amatoxin-linker conjugate, or derivative thereof, and the amatoxin-linker conjugate is represented by formula IIIB, wherein

R₁ is H, OH, OR_{A}, or OR_{C};

R₂ is H, OH, OR_{B}, or OR_{C};

R_{A} and R_{B}, when present, together with the oxygen atoms to which they are bound, combine to form a 5-membered heterocycloalkyl of formula:

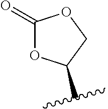

wherein R₃ is H or R_{C}.

In some embodiments, the cytotoxin is an amatoxin or derivative thereof and the amatoxin-linker conjugate is represented by formula IIIB, wherein

R₁ is H, OH, OR_{A}, or OR_{C};

R₂ is H, OH, OR_{B}, or OR_{C};

R_{A} and R_{B}, when present, together with the oxygen atoms to which they are bound, combine to form a 5-membered heterocycloalkyl group of formula:

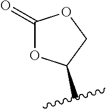

wherein

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH; and

wherein R_{C} and R_{D} are as defined above.

In some embodiments, the cytotoxin is an amatoxin or derivative thereof, and the amatoxin-linker conjugate is represented by formula IIIB, wherein:

R₁ is H, OH, or OR_{A};

R₂ is H, OH, or OR_{B};

R_{A} and R_{B}, when present, together with the oxygen atoms to which they are bound, combine to form a 5-membered heterocycloalkyl group of formula:

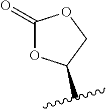

wherein

R₃, R₄, R₆, and R₇ are each H;

R₈ is OR_{C};

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2016/0002298, the disclosure of which is incorporated herein by reference in its entirety.

In some embodiments, the cytotoxin is an amatoxin or derivative thereof, and the amatoxin-linker conjugate is represented by formula IIIB, wherein:

R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2014/0294865, the disclosure of which is incorporated herein by reference in its entirety.

In some embodiments, the cytotoxin is an amatoxin or derivative thereof, and the amatoxin-linker conjugate is represented by formula IIIB, wherein:

R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH;

Q is —S—, —S(O)—, or —SO₂—; and

wherein R_{C} and R_{D} are as defined above. Such amatoxin-linker conjugates are described, for example, in U.S. Patent Application Publication No. 2015/0218220, the disclosure of which is incorporated herein by reference in its entirety.

In some embodiments, the cytotoxin is an amatoxin or derivative thereof, and the amatoxin-linker conjugate is represented by formula IIIB, wherein:

R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH;

Q is —S—, —S(O)—, or —SO₂—; and

wherein R_{C} and R_{D} are as defined above. Such amatoxin-linker conjugates are described, for example, in U.S. Pat. Nos. 9,233,173 and 9,399,681, the disclosures of each of which are incorporated herein by reference in their entirety.

Auristatins

Anti-CD2 antibodies and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF (MMAE and MMAF, respectively), disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE:

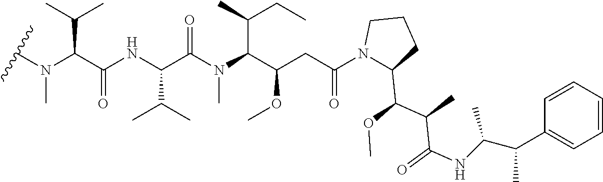 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](C)c1ccccc1)OC)C(C)C)C(C)C]

wherein the wavy line indicates the point of covalent attachment to the linker of an antibody-drug or drug-linker conjugate (-L-Z-Ab or -L-Z′, as described herein).

Another exemplary auristatin embodiment is MMAF,

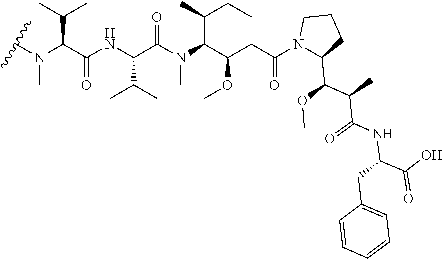 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

wherein the wavy line indicates the point of covalent attachment to the linker of an antibody-linker conjugate (-L-Z-Ab or -L-Z′, as described herein), as disclosed in US 2005/0238649:

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N²′-deacetyl-N²′-(3-mercapto-1-oxopropyl)-maytansine, as the cytotoxic agent. DM1 is represented by the following structural formula IV:

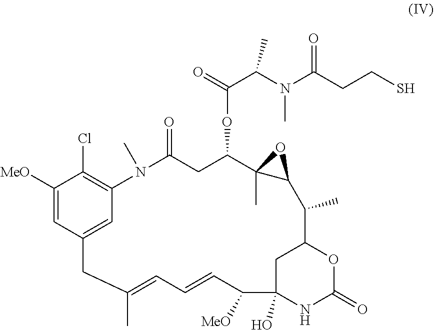 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present invention utilize the thiol-containing maytansinoid N²′-deacetyl-N²′(4-methyl-4-mercapto-1-oxopentyl)-maytansine (e.g., DM4) as the cytotoxic agent. DM4 is represented by the following structural formula V:

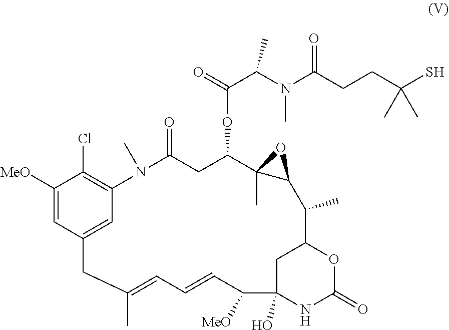 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N²′-deacetyl-N-²′(4-mercapto-1-oxopentyl)-maytansine (termed DM3), represented by the following structural formula VI:

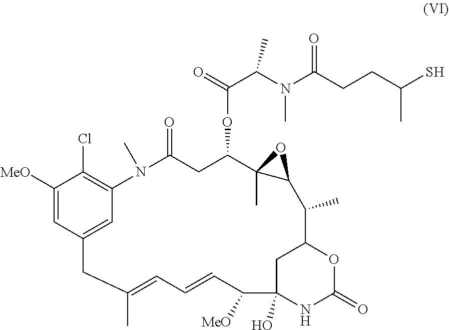 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugates of the present disclosure. In this regard, the entire disclosure of U.S. Pat. Nos. 5,208,020 and 7,276,697 is incorporated herein by reference.

Many positions on maytansinoids can serve as the position to covalently bond the linking moiety and, hence the antibodies or antigen-binding fragments thereof (-L-Z-Ab or -L-Z′, as described herein). For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to covalently bond the linker moiety, and in some particular embodiments, the C-3 position of maytansinol serves as the position to covalently bond the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present invention also includes various isomers and mixtures of maytansinoids and conjugates. Certain compounds and conjugates of the present invention may exist in various stereoisomeric, enantiomeric, and diastereomeric forms. Several descriptions for producing such antibody-maytansinoid conjugates are provided in U.S. Pat. Nos. 5,208,020; 5,416,064; 6,333,410; 6,441,163; 6,716,821; and 7,368,565, each of which is incorporated herein in its entirety.

Anthracyclines In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy: N. R. Bachur, “Free Radical Damage” id. at pp. 97-102]. Because of their cytotoxic potential anthracyclines have been used in the treatment of numerous cancers such as leukemia, breast carcinoma, lung carcinoma, ovarian adenocarcinoma and sarcomas [see e.g., P. H-Wiernik, in Anthracycline: Current Status And New Developments p 11]. Commonly used anthracyclines include doxorubicin, epirubicin, idarubicin and daunomycin.

Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.)

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by the structural formula:

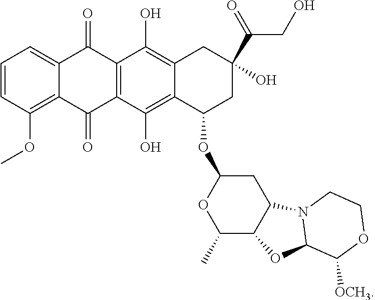

Multiple positions on anthracyclines such as PNU can serve as the position to covalently bond the linking moiety and, hence the anti-CD2 antibodies or antigen-binding fragments thereof as described herein. For example, linkers may be introduced through modifications to the hydroxymethyl ketone side chain.

In some embodiments, the cytotoxin is a PNU derivative represented by the structural

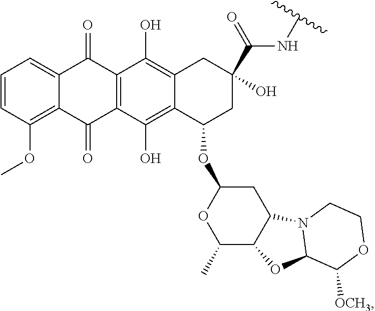

wherein the wavy line indicates the point of covalent attachment to the linker of the ADC as described herein.

Pyrrolobenzodiazepines (PBDs)

In other embodiments, the anti-CD2 antibodies or antigen-binding fragments thereof described herein can be conjugated to a cytotoxin that is a pyrrolobenzodiazepine (PBD) or a cytotoxin that comprises a PBD. PBDs may be produced by certain actinomycetes and have been shown to be sequence selective DNA alkylating compounds. PBD cytotoxins include, but are not limited to, anthramycin, dimeric PBDs, and those disclosed in, for example, Hartley, J A (2011) The development of pyrrolobenzodiazepines as antitumour agents. Expert Opin Inv Drug, 20(6), 733-744 and Antonow D, Thurston D E (2011) Synthesis of DNA-interactive pyrrolo[2,1-c][1,4]benzodiazepines (PBDs). Chem Rev 111: 2815-2864.

In some embodiments, the cytotoxin may be a pyrrolobenzodiazepine dimer represented by the structural formula:

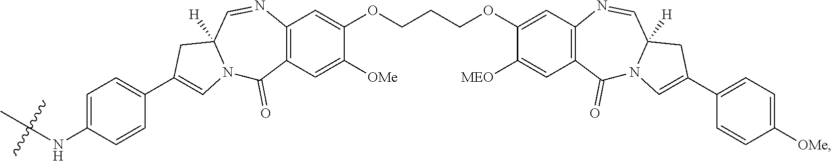

wherein the wavy line indicates the point of covalent attachment to the linker of the ADC as described herein. ADCs based on this PBD are disclosed in, for example, Sutherland et al., Blood 2013 122:1455-1463, which is incorporated by reference herein in its entirety.

In some embodiments, the cytotoxin may be a PBD dimer represented by the structural formula:

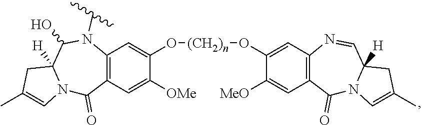

wherein n is 3 or 5, and wherein the wavy line indicates the point of covalent attachment to the linker of the ADC as described herein.

In some embodiments, the cytotoxin may be a PBD dimer represented by the structural formula:

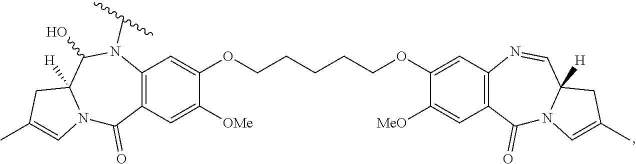

wherein the wavy line indicates the point of covalent attachment to the linker of the ADC as described herein.

In a specific embodiment, the cytotoxin may be a PBD dimer, which, when taken together with a linker and a reactive moiety Z′, each as described herein, may be represented by the structure:

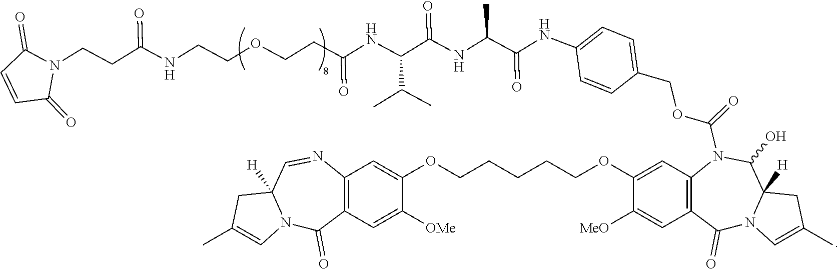

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In a specific embodiment, the cytotoxin may be a PBD dimer, which, when taken together with a linker and a reactive moiety Z′, each as described herein, may be represented by the structure:

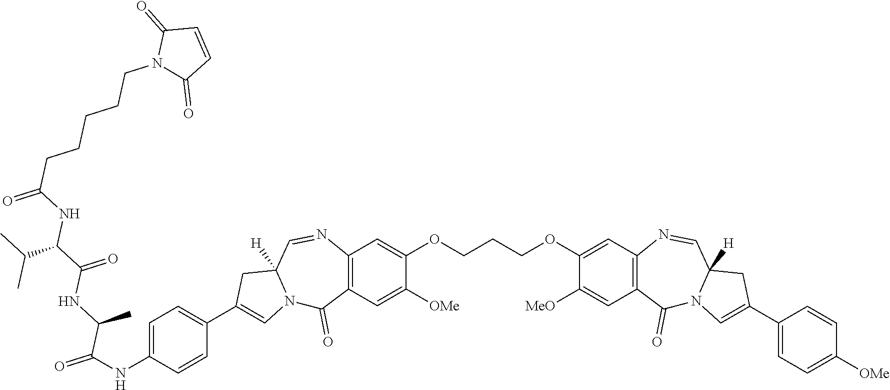 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in Mantaj et al., Angewandte Chemie International Edition English 2017,56, 462-488, the disclosure of which is incorporated by reference herein in its entirety.

Calicheamicin

In other embodiments, the antibodies and antigen-binding fragments thereof described herein can be conjugated to a cytotoxin that is an enediyne antitumor antibiotic (e.g., calicheamicins, ozogamicin).

The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

An exemplary calicheamicin is designated 71, which is herein referenced simply as gamma, and has the structural formula:

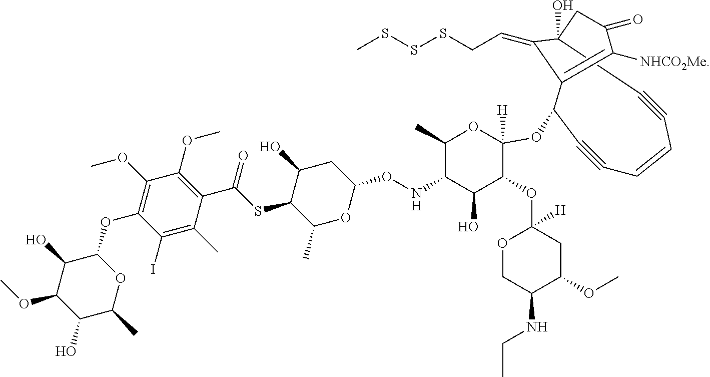

In some embodiments, the calicheamicin is a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD137 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein is a calicheamicin disulfide derivative represented by the formula:

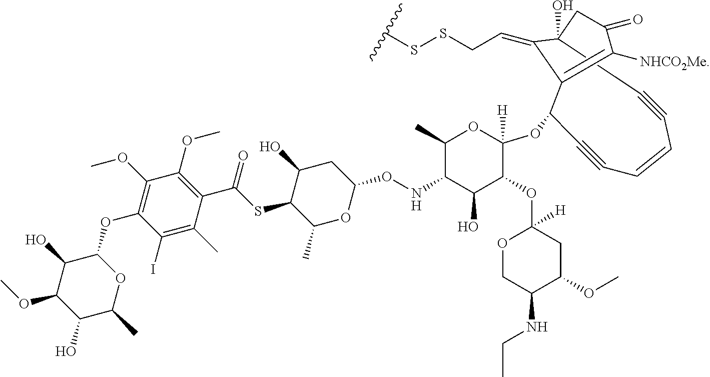

wherein the wavy line indicates the attachment point of the linker.

Additional Cytotoxins

In other embodiments, the antibodies and antigen-binding fragments thereof described herein can be conjugated to a cytotoxin other than or in addition to those cytotoxins disclosed herein above. Additional cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

### Linkers

The term “Linker” as used herein means a divalent chemical moiety comprising a covalent bond or a chain of atoms that covalently attaches an anti-CD2 antibody or fragment thereof (Ab) to a drug moiety (D) to form antibody-drug conjugates (ADC) of formula I. Suitable linkers have two reactive termini, one for conjugation to an antibody and the other for conjugation to a cytotoxin. The antibody conjugation reactive terminus of the linker (reactive moiety, Z′) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the cytotoxin conjugation reactive terminus of the linker is typically a site that is capable of conjugation to the cytotoxin. Non-limiting examples for linker-cytotoxin conjugation include, for example, formation of an amide bond with a basic amine or carboxyl group on the cytotoxin, via a carboxyl or basic amine group on the linker, respectively, or formation of an ether or the like, via alkylation of an OH group on the cytotoxin, via e.g., a leaving group on the linker. In some embodiments, cytotoxin-linker conjugation is through formation of an amide bond with a basic amine or carboxyl group on the cytotoxin, and so the reactive substituent on the linker is respectively a carboxyl or basic amine group. When the term “linker” is used in describing the linker in conjugated form, one or both of the reactive termini will be absent (such as reactive moiety Z′, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

A variety of linkers can be used to conjugate the antibodies, antigen-binding fragments, and ligands described to a cytotoxic molecule. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine.

Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, antigen-binding fragments, and ligands described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the aforementioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N.

In some embodiments, each C₁-C₆ alkyl, C₁-C₆ heteroalkyl, C₂-C₆ alkenyl, C₂-C₆ heteroalkenyl, C₂-C₆ alkynyl, C₂-C₆ heteroalkynyl, C₃-C₆ cycloalkyl, heterocycloalkyl, aryl, or heteroaryl group may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

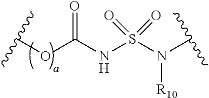

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a dipeptide selected from the group consisting of Phe-Lys, Val-Lys, Phe-Ala, Phe-Cit, Val-Ala, Val-Cit, and Val-Arg. In some embodiments, the linker comprises one or more of PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₚ— unit, wherein p is an integer from 1-6.

In some embodiments, the linker comprises a —(CH)₂ₙ— unit, where n is an integer from 2-6. In some embodiments, the linker includes —((CH₂)ₙ where n is 6. In some embodiments, L-Z is

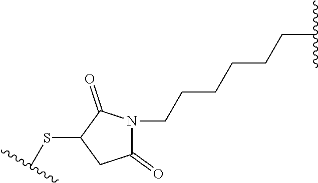

where S is a sulfur atom which represents the reactive substituent present within an antibody, or antigen-binding fragment thereof, that binds CD2 (e.g., from the —SH group of a cysteine residue).

In some embodiments, the linker comprises a ((CH₂)ₘO)ₙ(CH₂)ₘ— group where n and m are each independently selected from 1, 2, 3, 4, 5, 6, 7, 8, 9, and 10; and a heteroaryl group, wherein the heteroaryl group is a triazole. In some embodiments, the ((CH₂)ₘO)ₙ(CH₂)ₘ— group and triazole together comprise

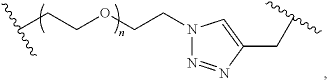

where n is from 1 to 10, and the wavy lines indicate attachment points to additional linker components, the chemical moiety Z, or the amatoxin. Other linkers that may used in the methods and compositions described herein are described in US 2019/0144504, which is incorporated by reference herein.

In one specific embodiment, the linker comprises PAB-Ala-Val-propionyl, represented by the structure

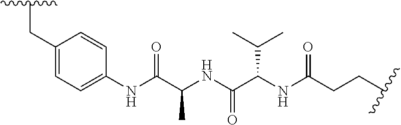 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises PAB-Cit-Val-propionyl, represented by the structure

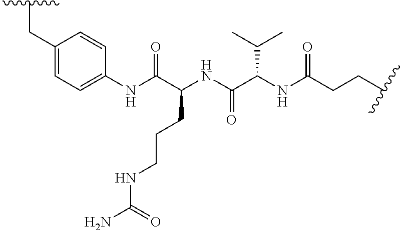 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z′. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 2, below. Curved lines designate points of attachment to the antibody or antigen-binding fragment, and the cytotoxic molecule, respectively.

TABLE 2
Exemplary chemical moieties Z formed by coupling reactions in the formation of
antibody-drug conjugates

| Exemplary | |
|---|---|
| Coupling Reactions | Chemical Moiety Z Formed by Coupling Reactions |
| [3 + 2] Cycloaddition | 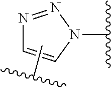 |
| [3 + 2] Cycloaddition | 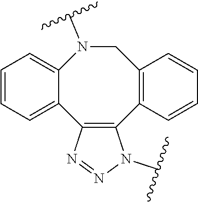 [SMILES: *N1Cc2ccccc2-c2c(nnn2*)-c2ccccc21] |
| [3 + 2] Cycloaddition, Esterification | 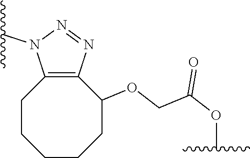 [SMILES: *OC(=O)COC1CCCCCc2c1nnn2*] |
| [3 + 2] Cycloaddition, Esterification | 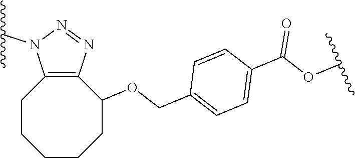 [SMILES: *OC(=O)c1ccc(COC2CCCCCc3c2nnn3*)cc1] |
| [3 + 2] Cycloaddition, Esterification | 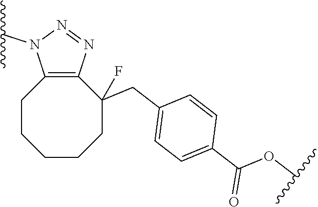 [SMILES: *OC(=O)c1ccc(CC2(F)CCCCCc3c2nnn3*)cc1] |
| [3 + 2] Cycloaddition, Esterification | 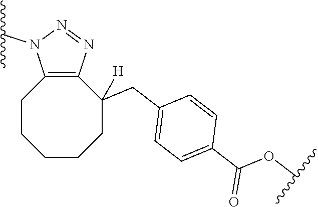 [SMILES: *OC(=O)c1ccc(CC2CCCCCc3c2nnn3*)cc1] |
| [3 + 2] Cycloaddition, Esterification | 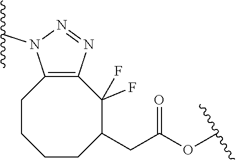 [SMILES: *OC(=O)CC1CCCCc2c(nnn2*)C1(F)F] |
| [3 + 2] Cycloaddition, Esterification | 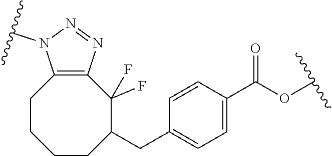 [SMILES: *OC(=O)c1ccc(CC2CCCCc3c(nnn3*)C2(F)F)cc1] |
| [3 + 2] Cycloaddition, Esterification | 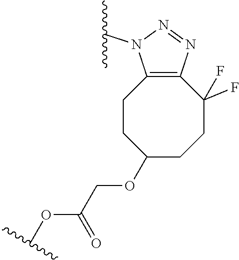 [SMILES: *OC(=O)COC1CCc2c(nnn2*)C(F)(F)CC1] |
| [3 + 2] Cycloaddition, Esterification | 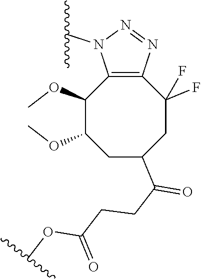 [SMILES: *OC(=O)CCC(=O)C1C[C@H](OC)[C@@H](OC)c2c(nnn2*)C(F)(F)C1] |
| [3 + 2] Cycloaddition, Esterification | 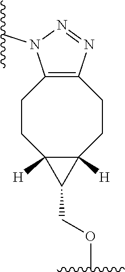 [SMILES: *OC[C@@H]1[C@@H]2CCc3c(nnn3*)CC[C@@H]21] |
| [3 + 2] Cycloaddition, Esterification | 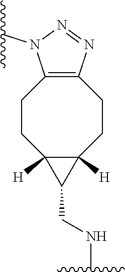 [SMILES: *NC[C@@H]1[C@@H]2CCc3c(nnn3*)CC[C@@H]21] |
| [3 + 2] Cycloaddition, Esterification | 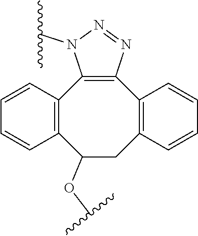 [SMILES: *OC1Cc2ccccc2-c2nnn(*)c2-c2ccccc21] |
| [3 + 2] Cycloaddition, Esterification | 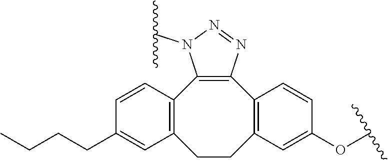 [SMILES: *Oc1ccc2c(c1)CCc1cc(CCCC)ccc1-c1c-2nnn1*] |
| [3 + 2] Cycloaddition | 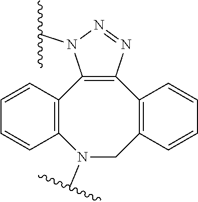 [SMILES: *N1Cc2ccccc2-c2nnn(*)c2-c2ccccc21] |
| Michael addition | 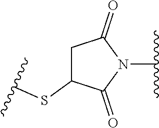 [SMILES: *SC1CC(=O)N(*)C1=O] |
| Michael addition | 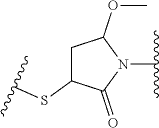 [SMILES: *SC1CC(OC)N(*)C1=O] |
| Imine condensation, Amidation | 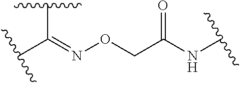 [SMILES: *NC(=O)CON=C(*)*] |
| Imine condensation | 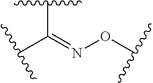 [SMILES: *ON=C(*)*] |
| Disulfide formation | 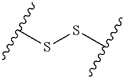 [SMILES: *SS*] |
| Thiol alkylation | 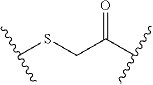 [SMILES: *SCC(*)=O] |
| Condensation, Michael addition | 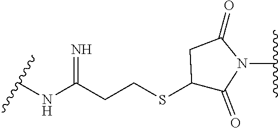 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

As depicted in Table 2, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others. Several representative and non-limiting examples of reactive substituents Z′ and the resulting chemical moieties Z are provided in Table 3.

TABLE 3
Complementary reactive substituents and chemical moieties

| | Functional Group on | | |
|---|---|---|---|
| | Antibody | Z′ group | Z group |
| Naturally Occurring | 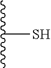 [SMILES: *S] | 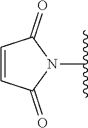 [SMILES: *N1C(=O)C=CC1=O] | 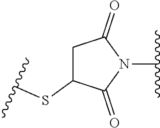 [SMILES: *SC1CC(=O)N(*)C1=O] |
| | | 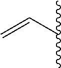 [SMILES: *C=C] | 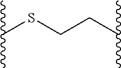 [SMILES: *CCS*] |
| | 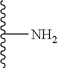 [SMILES: *N] | 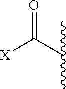 | 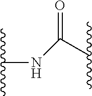 [SMILES: *NC(*)=O] |
| Synthetically Introduced | 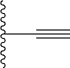 [SMILES: *C#C] | 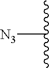 [SMILES: *N=[N+]=[N-]] | 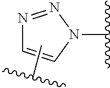 |
| | 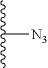 [SMILES: *N=[N+]=[N-]] | 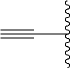 [SMILES: *C#C] | 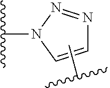 |
| | | 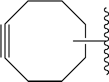 | 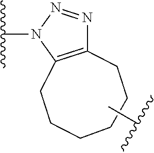 |
| | 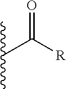 R = H or alkyl | 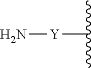 (Y = O or NH) | 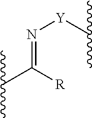 |

For instance, linkers suitable for the synthesis of drug-antibody and drug-ligand conjugates include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (hetero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

Non-limiting examples of amatoxin-linker conjugates containing a reactive substituent Z′ suitable for reaction with a reactive residue on the antibody or antigen-binding fragment thereof include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-1-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-lyl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyl)aziridin-1-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin.

In some embodiments, the chemical moiety Z is selected from Table 3 or Table 4. In some embodiments, the chemical moiety Z is:

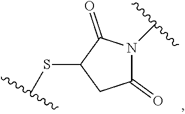

where S is a sulfur atom which represents the reactive substituent present within an antibody, or antigen-binding fragment thereof, such as an anti-CD5 antibody.

In some embodiments, an amatoxin as disclosed herein is conjugated to a linker-reactive moiety -L-Z′ having the following formula:

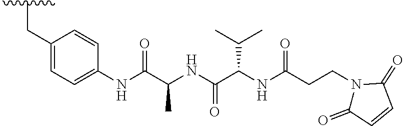 [SMILES: *Cc1ccc(NC(=O)[C@H](C)NC(=O)[C@@H](NC(=O)CCN2C(=O)C=CC2=O)C(C)C)cc1]

where the wavy line indicates the point of attachment to a substituent on the cytotoxin (e.g., an amatoxin). This linker-reactive substituent group L-Z′ may alternatively be referred to as N-beta-maleimidopropionyl-Val-Ala-para-aminobenzyl (BMP-Val-Ala-PAB).

In some embodiments, an amatoxin as disclosed herein is conjugated to a linker-reactive moiety -L-Z′ having the following formula:

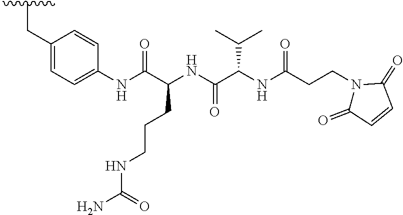 [SMILES: *Cc1ccc(NC(=O)[C@H](CCCNC(N)=O)NC(=O)[C@@H](NC(=O)CCN2C(=O)C=CC2=O)C(C)C)cc1]

where the wavy line indicates the point of attachment to a substituent on the cytotoxin (e.g., an amatoxin). This linker-reactive substituent group L-Z′ may alternatively be referred to as N-beta-maleimidopropyl-Val-Cit-para-aminobenzyl (BMP-Val-Cit-PAB).

In some embodiments, the linker L and the chemical moiety Z, taken together as L-Z, is

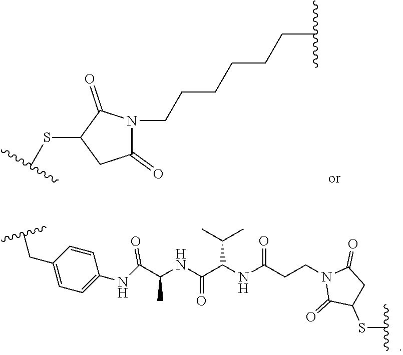

where S is a sulfur atom which represents the reactive substituent present within an antibody, or antigen-binding fragment thereof, such as an anti-CD5 antibody. The wavy line at the linker terminus indicates the point of attachment to the amatoxin.

In some embodiments, the linker L and the chemical moiety Z, after conjugation to the antibody, taken together as L-Z-Ab, has the structure:

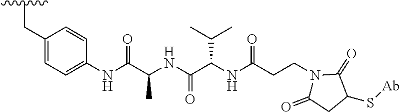

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In one embodiment, the CD2 antibodies, or antigen-binding fragments, described herein may be bound to an amatoxin so as to form a conjugate represented by the formula Ab-Z-L-Am, wherein Ab is the CD2 antibody, or antigen-binding fragment thereof, L is a linker, Z is a chemical moiety and Am is an amatoxin, each as described herein.

In some embodiments, Am-L-Z-Ab is:

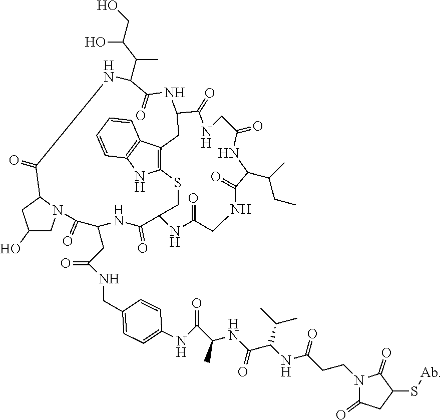

In some embodiments, Am-L-Z-Ab is:

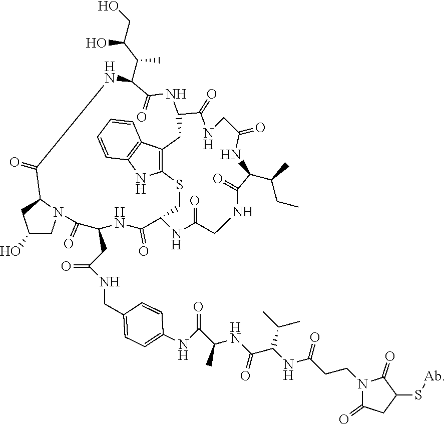

In some embodiments, Am-L-Z-Ab is:

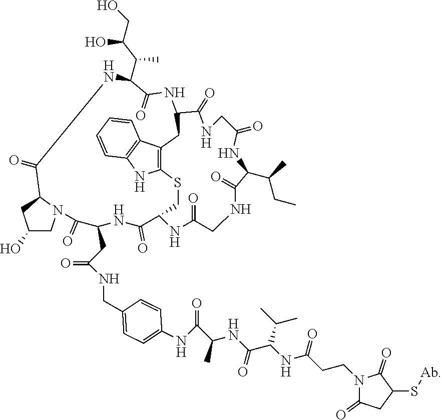

In some embodiments, Am-L-Z-Ab is:

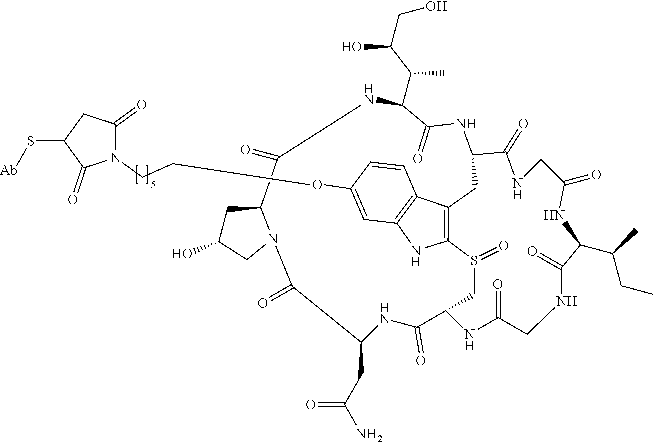

In some embodiments, Am-L-Z-Ab is:

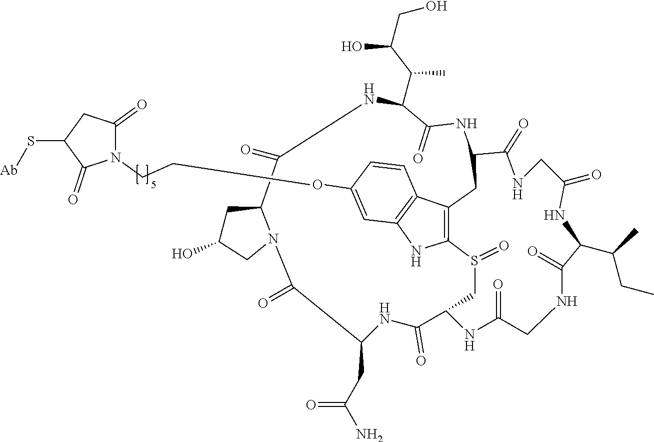

Preparation of Antibody-Drug Conjugates

In the ADCs of formula I as disclosed herein, an anti-CD2 antibody or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above. Additional methods for preparing ADC are described herein.

In another aspect, the anti-CD2 antibody or antigen binding fragment thereof has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-CD2 antibody or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the anti-CD2 antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

### Routes of Administration and Dosing

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising anti-CD2 ADCs as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The amount of ADC administered should be sufficient to deplete cells, e.g., activated T cells, which reject CAR cell therapy. The determination of a therapeutically effective dose is within the capability of practitioners in this art, however, as an example, in embodiments of the method described herein utilizing systemic administration of an ADC for the treatment of an immune disease or cancer, an effective human dose will be in the range of 0.1-150 mg/kg (e.g., 5 mg/kg, 10 mg/kg, 25 mg/kg, 50 mg/kg, 75 mg/kg, 100 mg/kg, 150 mg/kg etc.). The route of administration may affect the recommended dose. Repeated systemic doses are contemplated in order to maintain an effective level, e.g., to reduce the risk of CAR-T cell rejection, depending on the mode of administration adopted.

The anti-CD2 ADCs described herein may be administered by a variety of routes, such as orally, transdermally, subcutaneously, intranasally, intravenously, intramuscularly, intraocularly, or parenterally. The most suitable route for administration in any given case will depend on the particular ADC, the patient, pharmaceutical formulation methods, administration methods (e.g., administration time and administration route), the patient's age, body weight, sex, severity of the diseases being treated, the patient's diet, and the patient's excretion rate.

The effective dose of an anti-CD2 ADC described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the anti-CD2 ADC. A dose of the anti-CD2 ADC may be administered one or more times (e.g., 2-10 times) per day, week, or month to a human subject who has had, is concomitantly receiving, or will be receiving CAR therapy at a time point following delivery of the antu-CD2 ADC. An anti-CD2 ADC may be administered to the human patient one time or as multiple doses. In one embodiment, the anti-CD2 ADC can be administered in an amount sufficient to reduce the quantity of host-reactive T cells, for example, by 10%, 20%, 30%, 40%, 50%, 60%, 70%, 80%, 90%, 95%, or more prior to CAR therapy.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

The data in Examples 1-6 below are described in International Patent Application No. WO 2019/108860, which is incorporated by reference herein.

### Example 1: In Vitro Binding Analysis of Anti-CD2 Antibodies

To determine the binding characteristics of anti-CD2 antibodies RPA-2.10 mIgG1 and Abl hIgG1, antibody binding studies were performed at 25 degrees Celsius in lx PBS supplemented with 0.1% w/v bovine serum albumin with a Pall ForteBio Octet Red96 using biolayer interferometry (BLI). The indicated purified human (Abl-hIgG1) or murine (RPA-2.10 mIgG1) antibody was immobilized onto anti-human Fc biosensors (AHC; Pall ForteBio 18-5063) or anti-murine Fc biosensors (AMQ; Pall ForteBio 18-5090 and incubated with 50 nM of purified human CD2 ectodomain (Sigma Aldrich and Catalog #5086). The apparent monovalent affinity (K_{D}), apparent association rate (K_{ON}), and apparent dissociation rate (K_{DIS}) were determined by local full fitting with a 1:1 binding model as calculated by ForteBio data analysis software version 10 of each IgG to purified human CD2 ectodomain are shown in Table 3.

Further characterization of anti-CD2 antibodies is provided in Examples 2 to 6.

TABLE 3
Binding kinetics of the indicated IgG to human CD2 ectodomain

| | Conc. | Response | K_{D} | K_{ON} | K_{DIS} | Full |
|---|---|---|---|---|---|---|
| Antibody | (nM) | (nm) | (M) | (1/Ms) | (1/s) | R² |
| mRPA-2.10 | 50 | 0.1807 | 2.00E−09 | 8.60E+04 | 1.72E−04 | 0.9952 |
| Ab1 | 50 | 0.0615 | 2.12E−09 | 1.36E+05 | 2.89E−04 | 0.9683 |

### Example 2: In Vitro Cell Line Binding Analysis of Anti-CD2 Antibodies

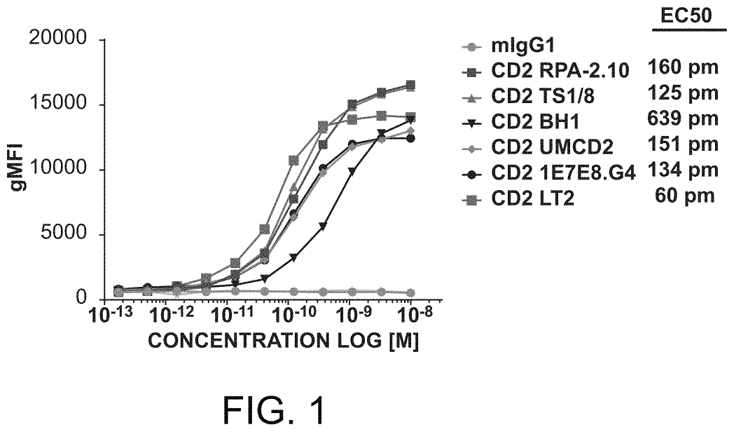

MOLT-4 cells (i.e., an immortalized human T lymphoblast cell line) were plated at 20,000 cells/well and stained with a titration of the indicated murine anti-CD2 antibodies (i.e., RPA-2.10, TS1/8, BH1, UMCD2, 1E7E8.G4, or LT2) for 2 hours at 4° C. Secondary anti-mouse AF488 stain, at a constant amount, was added for 30 minutes at 4° C. After washing, plates were run on a flow cytometer and binding of the indicated antibody (and the negative control, i.e., mIgG1) was determined based on geometric mean fluorescence intensity in the AF488 channel. Results from these assays are provided in FIG. 1.

As shown in FIG. 1, the murine anti-CD2 antibodies RPA-2.10, TS1/8, BH1, UMCD2, 1E7E8.G4, and LT2 bind to human T lymphoblast cells (i.e. MOLT-4 cells), with an EC₅₀=160 μM (RPA-2.10), 125 μM (TS 1/8), 639 μM (BH1), 151 μM (UMCD2), 134 μM (1E7E8), and 60 μM (LT2).

### Example 3: In Vitro Primary Cell Binding Analysis of Anti-CD2 Antibodies

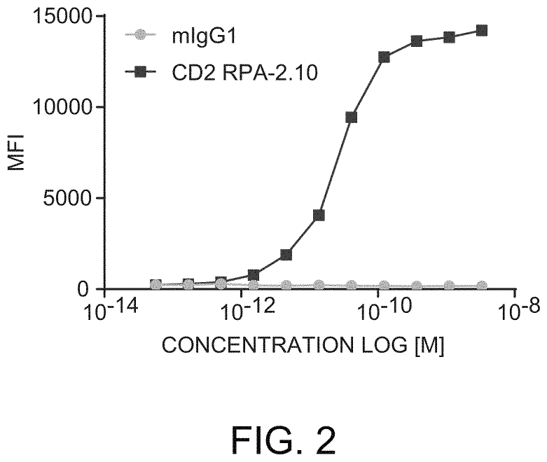

Primary human T-cells were plated at 8×10⁴ cells/well and stained with a titration of the murine anti-CD2 antibody RPA-2.10 for 2 hours at 37° C. Secondary anti-mouse or anti-human AF488 stain, relative to primary antibody, at a constant amount, was added for 30 minutes at 4° C. After washing, plates were run on a flow cytometer and binding of the indicated antibody (and the negative control, i.e., mIgG1 or hIgG1) was determined based on geometric mean fluorescence intensity in the AF488 channel. Results from these assays are provided in FIG. 2.

As shown in FIG. 2, the murine anti-CD2 antibody RPA-2.10 binds to primary human T-cells. with an EC₅₀=1.84 μM (RPA-2.10).

### Example 4. In Vitro Analysis of an Anti-CD2-Amanitin Antibody Drug Conjugate (ADC) Using an In Vitro T-Cell Killing Assay

The anti-CD2 antibody RPA 2.10 was conjugated to amanitin with a cleavable linker to form an anti-CD2-ADC. One anti-CD2-ADC was prepared from the murine anti-CD2 antibody RPA-2.10 having an average interchain drug-to-antibody ratio (DAR) of 6. A second anti-CD2-ADC having an average DAR of 2 was prepared using a human chimeric variant of RPA-2.10 conjugated to amanitin using site-specific conjugation. Further, a fast half-life variant of anti-CD2-ADC was generated through the introduction of a H435A mutation. Each anti-CD2-ADC was assessed using an in vitro T-cell killing assay.

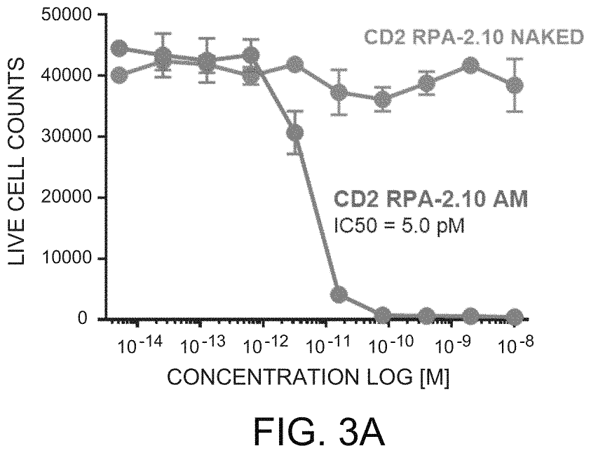

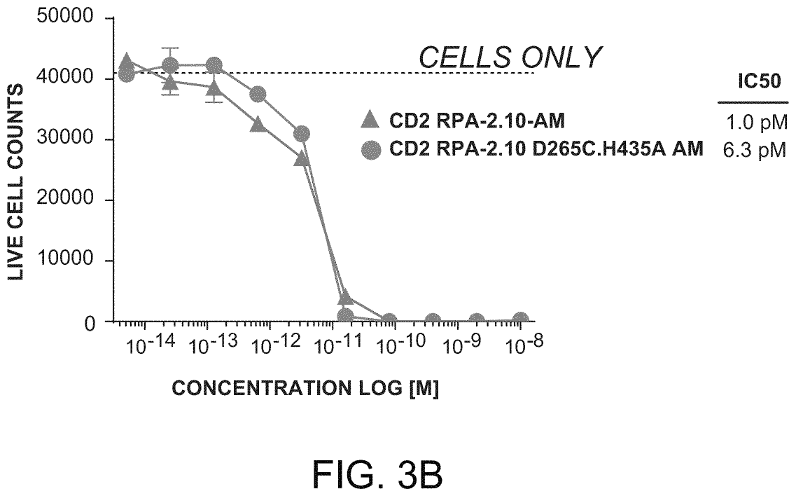

Cryopreserved negatively-selected primary human T cells were thawed and stimulated with anti-CD3 antibodies and IL-2. At the start of the assay, 2×10⁴ T cells were seeded per well of a 384 well plate and the indicated ADCs or non-conjugated anti-CD2 antibody were added to the wells at various concentrations between 0.003 nm and 30 nm before being placed in an incubator with 37° C. and 5% CO₂. Following five days of culture, cells were analyzed by flow cytometry. Cells were stained with a viability marker 7-AAD and run on a volumetric flow cytometer. Numbers of viable T-cells (FIGS. 3A and 3B) were determined by FSC vs SSC and 7-AAD staining. A non-conjugated anti-CD2 antibody (RPA 2.10) served as a comparator (FIG. 3A).

As shown in FIG. 3A, anti-CD2-ADCs having an interchain drug-to-antibody ratio of 6 exhibited potent and specific killing of T cells (IC50=5.0 pm) whereas T cells remained viable in the presence of non-conjugated (“naked”) anti-CD2 antibodies. As shown in FIG. 3B, human chimeric anti-CD2-ADCs having a site-specific drug-to-antibody ratio of 2 retained a potent level of T-cell killing (IC50=1.0 pm) similar to that of the DAR 6 ADCs. Further, the fast-half life variant of the anti-CD2-ADCs (H435A) exhibited a similar level of T-cell killing (IC50=6.3 pm; FIG. 3B) as an anti-CD2-ADC with WT half-life.

### Example 5. In Vitro Analysis of an Anti-CD2-Amanitin Antibody Drug Conjugate (ADC) Using an In Vitro T-Cell Killing Assay

The anti-CD2 antibody RPA 2.10 was conjugated to amanitin with a cleavable linker to form an interchain anti-CD2-ADC with an average interchain drug-to-antibody ratio (DAR) of 6. The anti-CD2-ADC was assessed using an in vitro natural killer (NK)-cell killing assay.

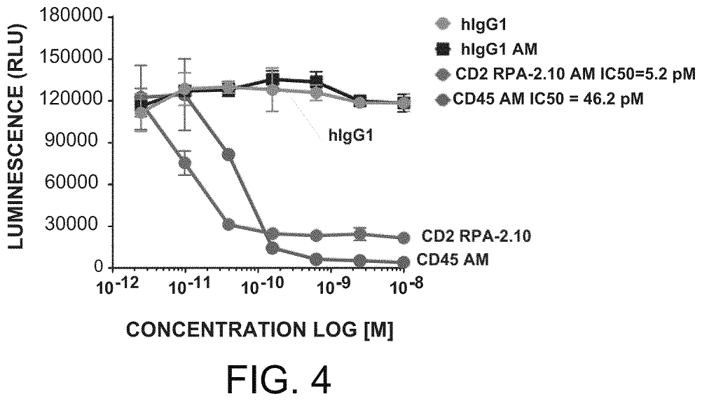

Primary human CD56+CD3-NK cells were cultured with recombinant IL-2 and IL-15 for four days. At the start of the assay, 30,000 freshly isolated NK cells from a healthy human donor were seeded per well of a 384 well plate and the indicated ADC or control (i.e., IgG1 or IgG1-amanitin ADC) was added to the wells at various concentrations between 0.003 nm and 30 nm before being placed in an incubator with 37° C. and 5% CO₂. Following 4 days of culture, NK cell viability was analyzed by a CellTiter-Glo assay (FIG. 4).

As shown in FIG. 4, anti-CD2-ADC exhibited potent killing of NK cells, with an IC50 of 5.2 pM. The lack of complete killing by the anti-CD2-ADC is consistent with the fact that CD2 is only expressed on about 75% of NK cells.

### Example 6. Analysis of T-Cell Depletion Using a hNSG Mouse Model

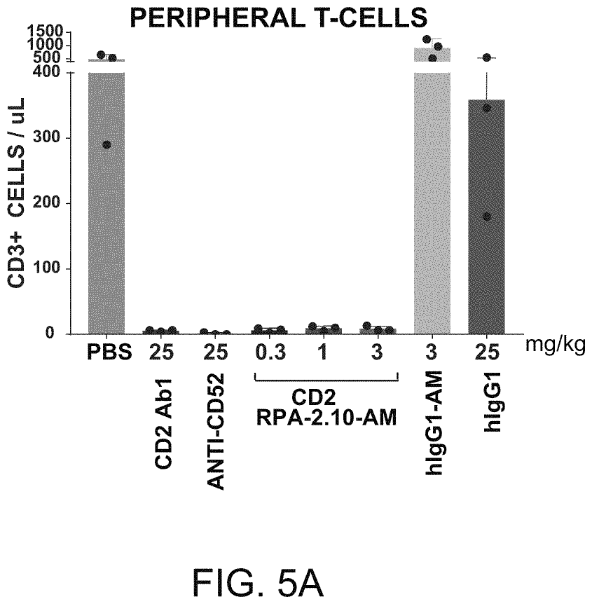

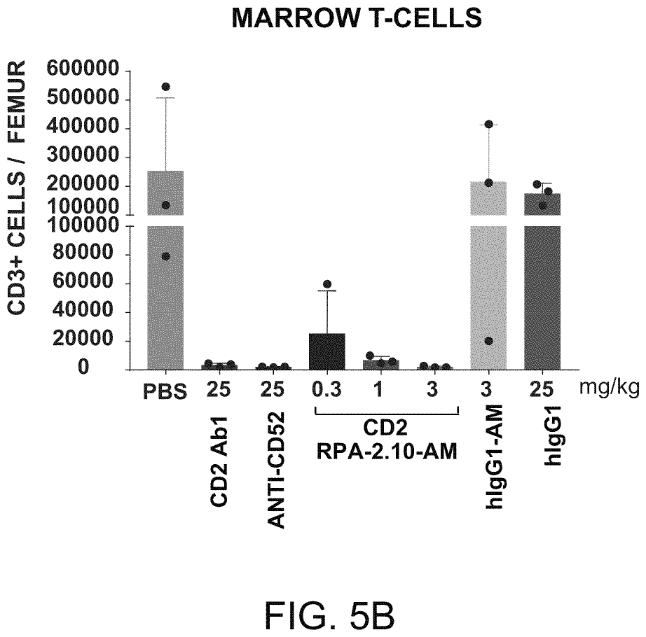

In vivo T-cell depletion assays were conducted using humanized NSG mice (Jackson Laboratories). An anti-CD2 antibody RPA 2.10 was conjugated to amanitin with a cleavable linker to form an anti-CD2-ADC. One anti-CD2-ADC was prepared with murine RPA 2.10 having an average interchain drug-to-antibody ratio (DAR) of 6 while another anti-CD2-ADC was prepared with human chimeric RPA 2.10 having an average site-specific DAR of 2. Each anti-CD2-ADC (DAR6 and DAR2) was administered as a single intravenous injection (0.3 mg/kg, 1 mg/kg, or 3 mg/kg for DAR6 ADCs, and 1 mg/kg or 3 mg/kg for DAR2 ADCs) to the humanized mouse model. Peripheral blood cells, bone marrow, or thymic samples were collected on Day 7 and the absolute number of CD3+ T-cells was determined by flow cytometry (see FIGS. 5A and 5B for DAR2 ADCs, and 6A-6C for DAR6 ADCs).

As shown in, FIGS. 5A-5B, humanized NSG mice treated with 0.3 mg/kg, 1 mg/kg, or 3 mg/kg interchain DAR6 anti-CD2-ADC exhibited potent T-cell depletion in peripheral blood or bone morrow while thymic T-cells were depleted following treatment with 3 mg/kg of DAR6 anti-CD2-ADC. For comparison, FIGS. 5A and 5B also show the level of T-cell depletion following treatment of humanized NSG mice with 25 mg/kg Abl (an unconjugated anti-CD2 antibody) or with the indicated controls (i.e., 25 mg/kg anti-CD52 antibody (clone YTH34.5); 3 mg/kg hIgG1-amanitan ADC (“hIgG1-AM”), 25 mg/kg hIgG1, or PBS).

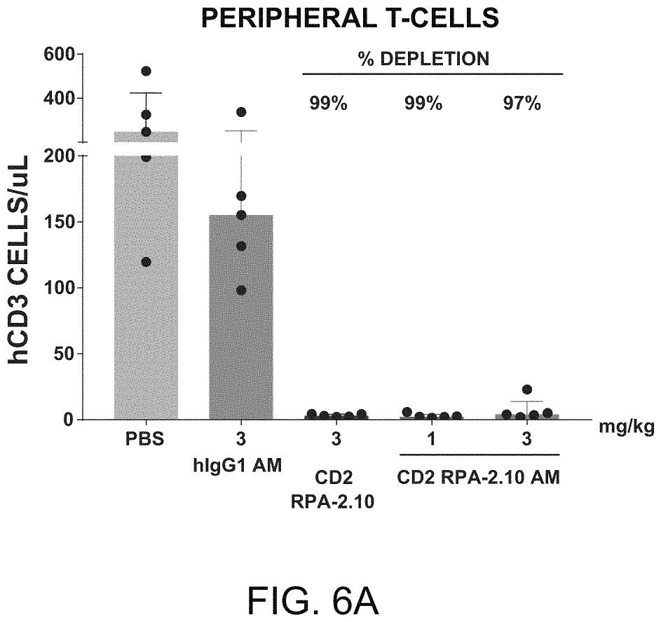

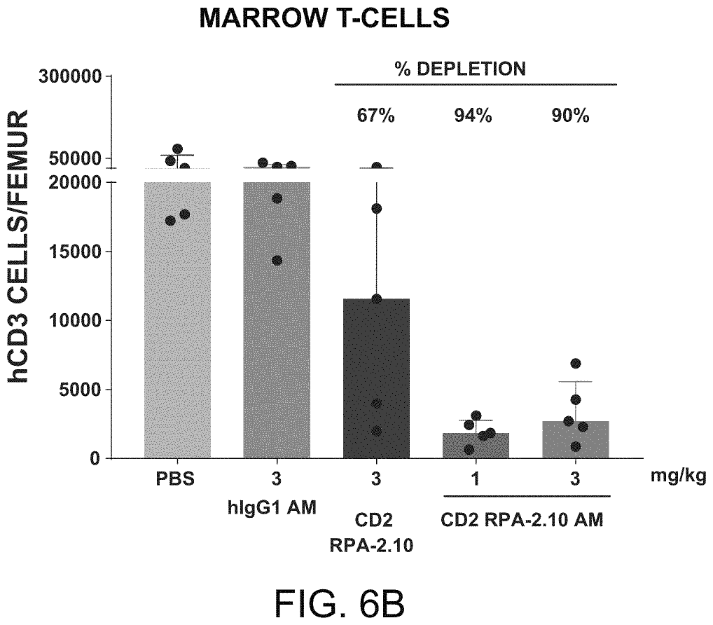

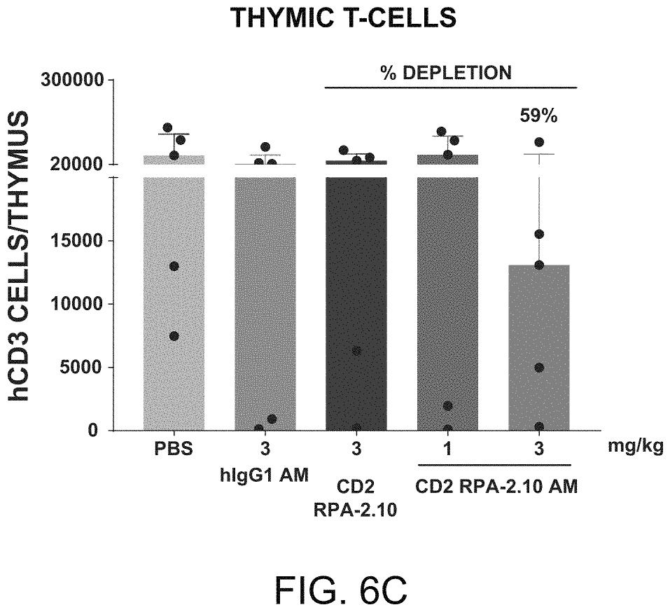

As shown in, FIGS. 6A-6C, humanized NSG mice treated with 1 mg/kg or 3 mg/kg site-specific DAR2 anti-CD2-ADC exhibited potent T-cell depletion in peripheral blood or bone morrow while thymic T-cells displayed about 59% depleted following treatment with 3 mg/kg of DAR2 anti-CD2-ADC. For comparison, FIGS. 6A-6C also show the level of T-cell depletion following treatment of humanized NSG mice with 3 mg/kg of an unconjugated anti-CD2 antibody or with the indicated controls (i.e., 3 mg/kg hIgG1-amanitan-ADC (“hIgG1-AMC”) or PBS).

### Example 7 Administration of Allogeneic CAR-T Cells in a Mouse Model

The following study is performed to assess the level of CAR-T cells present in an allogeneic recipient under different conditions.

A murine allogeneic CAR-T model is used for this study.

A first treatment group of mice is treated with a priming dose of allogeneic T cells, by administration of 1×10⁷ to 1×10⁹ cells/kg by intravenous infusion at Day 0. On Day 3, the mice are administered an anti-CD2-α-amanitin ADC at a dose of 3 mg/kg. On Day 10, after the ADC has substantially cleared from the blood of the mice, mice are administered allogeneic CAR-T cells. The CAR-T cells are from the same donor as the allogeneic T cells administered on Day 0.

A second treatment group of mice is treated using the same protocol as the first treatment group, but is administered an unconjugated anti-CD2 antibody on Day 3, in place of the anti-CD2 ADC.

A third treatment group of mice is treated using the same protocol as the first treatment group, but is administered an isotype control antibody conjugated to α-amanitin on Day 3, in place of the anti-CD2 ADC.

A fourth treatment group of mice is treated using the same protocol as the first treatment group, but is administered a priming dose of autologous T cells at Day 0, in place of the allogeneic T cells.

A fifth treatment group of mice is administered allogeneic CAR-T cells at Day 10, without prior treatment.

A sixth treatment group of mice is administered autologous CAR-T cells at Day 10, without prior treatment.

The number of CAR-T cells present in spleen and peripheral blood of mice from each treatment group is determined at Day 14, Day 17, and Day 30. The number of CD2+ activated T cells in the spleen and peripheral blood of mice from each treatment group is determined at Day 9. Mice are monitored for symptoms of rejection and presence of CAR-T cells throughout the study.

### Example 8: Administration of an Anti-CD2 Antibody Drug Conjugate to a Human

Patient to Prevent Rejection of an Allogeneic Cell Therapy A human patient is selected to receive an allogeneic cell therapy, such as an allogeneic CAR cell therapy. To inhibit or prevent the rejection of the allogeneic cells, an anti-CD2 antibody drug conjugate (ADC) is administered in accordance with the methods disclosed herein. The physician carries out the following treatment steps.

First, an initial amount of an allogeneic cell is intravenously administered to the patient in an amount sufficient to elicit a priming immune response to the allogeneic cell. In the priming step, allogeneic cells are administered to the patient to elicit an immune response resulting in endogenous activated CD2+ T cells.

Subsequently, the patient is administered an anti-CD2 ADC comprising an anti-CD137 antibody conjugated to a cytotoxin via a linker. The anti-CD2 ADC is administered in an amount effective to deplete endogenous CD2+ activated T cells. The level of CD2+ activated T cells is assessed in the patient following administration of the anti-CD2 ADC to confirm depletion.

Next, the patient is administered a therapeutically effective amount of allogeneic cells expressing a CAR. The allogeneic cells are derived from the same donor as the cells administered to the patient during the priming step. Acceptance of the allogeneic cells in the recipient patient is promoted and the risk of rejection is reduced, relative to patients receiving an allogeneic cell therapy without priming and administration of an anti-CD2 ADC.

TABLE 4
Sequence Summary

| Sequence | | |
|---|---|---|
| Identifier | Description | Sequence |
| SEQ ID NO: 19 | Ab1 CDR-H1 | EYYMY |
| SEQ ID NO: 20 | Ab1 CDR-H2 | RIDPEDGSIDYVEKFKK |
| SEQ ID NO: 21 | Ab1 CDR-H3 | GKFNYRFAY |
| SEQ ID NO: 22 | Ab1 CDR-L1 | RSSQSLLHSSGNTYLN |
| SEQ ID NO: 23 | Ab1 CDR-L2 | LVSKLES |
| SEQ ID NO: 24 | Ab1 CDR-L3 | MQFTHYPYT |
| SEQ ID NO: 25 | Ab1 Heavy chain | QVQLVQSGAEVKKPGASVKVSCKASGYTFTEYY |
| | variable region | MYWVRQAPGQGLELMGRIDPEDGSIDYVEKFKK |
| | | KVTLTADTSSSTAYMELSSLTSDDTAVYYCARGK |
| | | FNYRFAYWGQGTLVTVSS |
| SEQ ID NO: 26 | Ab1 Light chain | DVVMTQSPPSLLVTLGQPASISCRSSQSLLHSSG |
| | variable region | NTYLNWLLQRPGQSPQPLIYLVSKLESGVPDRFS |
| | | GSGSGTDFTLKISGVEAEDVGVYYCMQFTHYPYT |
| | | FGQGTKLEIK |
| SEQ ID NO: 27 | Ab1 a Heavy chain | QVQLVQSGAEVQRPGASVKVSCKASGYIFTEYY |
| | variable region | MYWVRQAPGQGLELVGRIDPEDGSIDYVEKFKKK |
| | | VTLTADTSSSTAYMELSSLTSDDTAVYYCARGKF |
| | | NYRFAYWGQGTLVTVSS |
| SEQ ID NO: 28 | Ab1 a Light chain | DVVMTQSPPSLLVTLGQPASISCRSSQSLLHSSG |
| | variable region | NTYLNWLLQRPGQSPQPLIYLVSKLESGVPDRFS |
| | | GSGSGTDFTLKISGVEAEDVGVYYCMQFTHYPYT |
| | | FGQGTKLEIK |
| SEQ ID NO: 29 | Consensus human | EVQLVESGGGLVQPGGSLRLSCAASGFTFSDYA |
| | Ab | MSWVRQAPGKGLEWVAVISENGSDTYYADSVKG |
| | Heavy chain variable | RFTISRDDSKNTLYLQMNSLRAEDTAVYYCARDR |
| | domain | GGAVSYFDVWGQGTLVTVSS |
| SEQ ID NO: 30 | Consensus human | DIQMTQSPSSLSASVGDRVTITCRASQDVSSYLA |
| | Ab | WYQQKPGKAPKLLIYAASSLESGVPSRFSGSGS |
| | Light chain variable | GTDFTLTISSLQPEDFATYYCQQYNSLPYTFGQG |
| | domain | TKVEIKRT |
| SEQ ID NO: 31 | Human CD2 | MSFPCKFVAS FLLIFNVSSK GAVSKEITNA |
| | sequence | LETWGALGQD INLDIPSFQM SDDIDDIKWE |
| | | KTSDKKKIAQ FRKEKETFKE KDTYKLFKNG |
| | | TLKIKHLKTD DQDIYKVSIY DTKGKNVLEK |
| | | IFDLKIQERV SKPKISWICI NTTLTCEVMN |
| | | GTDPELNLYQ DGKHLKLSQR VITHKWTTSL |
| | | SAKFKCTAGN KVSKESSVEP VSCPEKGLDI |
| | | YLIIGICGGG SLLMVFVALL VFYITKRKKQ |
| | | RSRRNDEELE TRAHRVATEE RGRKPHQIPA |
| | | STPQNPATSQ HPPPPPGHRS QAPSHRPPPP |
| | | GHRVQHQPQK RPPAPSGTQV HQQKGPPLPR |
| | | PRVQPKPPHG AAENSLSPSS N |
| SEQ ID NO: 32 | RPA-2.10 CDR-H1 | GFTFSSY |
| SEQ ID NO: 33 | RPA-2.10 CDR-H2 | SGGGF |
| SEQ ID NO: 34 | RPA-2.10 CDR-H3 | SSYGEIMDY |
| | Variant 1 | |
| SEQ ID NO: 35 | RPA-2.10 CDR-H3 | SSYGELMDY |
| | Variant 2 | |
| SEQ ID NO: 36 | RPA-2.10 CDR-L1 | RASQRIGTSIH |
| SEQ ID NO: 37 | RPA-2.10 CDR-L2 | YASESIS |
| SEQ ID NO: 38 | RPA-2.10 CDR-L3 | QQSHGWPFTF |
| SEQ ID NO: 39 | RPA-2.10 Heavy | EVKLVESGGGLVKPGGSLKLSCAASGFTFSSYDM |
| | chain variable region | SWVRQTPEKRLEWVASISGGGFLYYLDSVKGRFT |
| | Variant 1 | ISRDNARNILYLHMTSLRSEDTAMYYCARSSYGEI |
| | | MDYWGQGTSVTVSS |
| SEQ ID NO: 40 | RPA-2.10 Heavy | EVKLVESGGGLVKPGGSLKLSCAASGFTFSSYDM |
| | chain variable region | SWVRQTPEKRLEWVASISGGGFLYYLDSVKGRFT |
| | Variant 2 | ISRDNARNILYLHMTSLRSEDTAMYYCARSSYGEL |
| | | MDYWGQGTSVTVSS |
| SEQ ID NO: 41 | RPA-2.10 Light | DILLTQSPAILSVSPGERVSFSCRASQRIGTSIHWY |
| | chain variable region | QQRTTGSPRLLIKYASESISGIPSRFSGSGSGTDF |
| | | TLSINSVESEDVADYYCQQSHGWPFTFGGGTKLE |
| | | IE |
| SEQ ID NO: 42 | RPA-2.10 Heavy | AKTTPPSVYPLAPGSAAQTNSMVTLGCLVKGYFP |
| | chain constant | EPVTVTWNSGSLSSGVHTFPAVLQSDLYTLSSSV |
| | region | TVPSSTWPSETVTCNVAHPASSTKVDKKIVPRDC |
| | | GCKPCICTVPEVSSVFIFPPKPKDVLTITLTPKVTC |
| | | VVVDISKDDPEVQFSWFVDDVEVHTAQTQPREE |
| | | QFNSTFRSVSELPIMHQDWLNGKEFKCRVNSAAF |
| | | PAPIEKTISKTKGRPKAPQVYTIPPPKEQMAKDKV |
| | | SLTCMITDFFPEDITVEWQWNGQPAENYKNTQPI |
| | | MDTDGSYFVYSKLNVQKSNWEAGNTFTCSVLHE |
| | | GLHNHHTEKSLSHSPGK |
| SEQ ID NO: 43 | RPA-2.10 Light | RADAAPTVSIFPPSSEQLTSGGASVVCFLNNFYPK |
| | chain constant | DINVKWKIDGSERQNGVLNSWTDQDSKDSTYSM |
| | region | SSTLTLTKDEYERHNSYTCEATHKTSTSPIVKSFN |
| | | RNEC |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

1. A method of promoting acceptance of an immune cell expressing a chimeric antigen receptor (CAR) in a human subject having cancer or an autoimmune disease, the method comprising
(a) administering an anti-CD2 antibody drug conjugate (ADC) to a human subject having cancer or an autoimmune disease, wherein the anti-CD2 ADC comprises an anti-CD2 antibody, or antigen-binding fragment thereof, conjugated to a cytotoxin via a linker; and
(b) administering a therapeutically effective amount of an immune cell expressing a CAR to the human subject, wherein the CAR comprises an extracellular domain that binds to a tumor antigen or an antigen associated with an autoimmune disease, a transmembrane domain, and a cytoplasmic domain.

2. The method of claim 1, wherein the human subject is not administered alemtuzumab and/or a lymphodepleting chemotherapeutic agent prior to, concomitantly with, or following step (b).

3. (canceled)

4. The method of claim 2, wherein the lymphodepleting chemotherapeutic agent is fludarabine, cyclophosphamide, bendamustine, and/or pentostatin.

5. The method of claim 1, further comprising administering an anti-CD2 ADC to the human subject prior to step (b).

6. (canceled)

7. The method of claim 1, wherein the immune cell is an allogeneic cell or an autologous cell.

8. The method of claim 7, wherein the allogeneic cell is an allogeneic T cell or an allogeneic NK cell.

9. (canceled)

10. A method of treating a patient having a tumor comprising (i) an anti-CD2 ADC, wherein the anti-CD2 ADC comprises an anti-CD2 antibody, or antigen-binding fragment thereof, conjugated to a cytotoxin via a linker, and (ii) administering to the patient a therapeutically effective amount of from about 1×10⁶ to about 1×10⁸ engineered CAR T cells/kg.

11. (canceled)

12. The method of claim 1, wherein the anti-CD2 ADC is administered to the patient as a single dose or as multiple doses.

13. The method of any one of claim 1, wherein the anti-CD2 antibody, or antigen-binding fragment thereof, comprises a heavy chain variable region comprising a CDR1, a CDR2, and a CDR3 having an amino acid sequence as set forth in SEQ ID NOs: 1, 2, and 3, respectively, and comprises a light chain variable region comprising a CDR1, a CDR2, and a CDR3 having an amino acid sequence as set forth in SEQ ID NOs: 4, 5, and 6, respectively.

14.-15. (canceled)

16. The method of claim 1, wherein the cytotoxin is an antimitotic agent or an RNA polymerase inhibitor.

17. The method of claim 16, wherein the RNA polymerase inhibitor is an amatoxin.

18.-19. (canceled)

20. The method of claim 17, wherein the anti-CD2 antibody, or antigen-binding fragment thereof conjugated to an amatoxin is represented by the formula Ab-Z-L-Am, wherein Ab is the antibody or antigen-binding fragment thereof, L is a linker, Z is a chemical moiety, and Am is an amatoxin represented by formula (III) 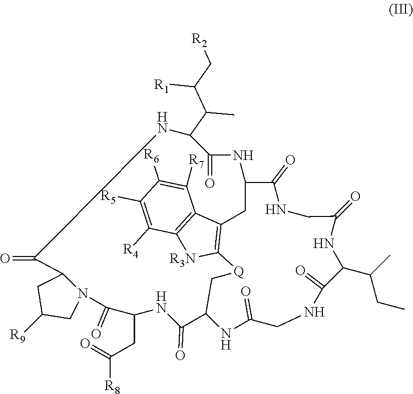
wherein R₁ is H, OH, OR_{A}, or OR_{C};
R₂ is H, OH, OR_{B}, or OR_{C};
R_{A} and R_{B}, when present, together with the oxygen atoms to which they are bound, combine to form an optionally substituted 5-membered heterocycloalkyl group;
R₃ is H, R_{C}, or R_{D};
R₄, R₅, R₆, and R₇ are each independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
X is —S—, —S(O)—, or —SO₂—;
R_{C} is -L-Z;
R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;
L is a linker; and
Z is a chemical moiety formed from a coupling reaction between a reactive substituent present on L and a reactive substituent present within the anti-CD2 antibody or antigen-binding fragment thereof, wherein Am comprises exactly one R_{c} substituent.

21. (canceled)

22. The method of claim 16, wherein the antimitotic agent is a maytansine, an auristatin, a pyrrolobenzodiazepine (PBD) or calicheamicin.

23.-28. (canceled)

29. The method of any one of claim 1, wherein the extracellular domain of the CAR comprises an scFv antibody or a single chain T cell receptor (scTCR).

30. (canceled)

31. The method of claim 1, wherein the tumor antigen is an antigen selected from the group consisting of CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

32. The method of claim 1, wherein the cytoplasmic domain of the CAR comprises a CD28 cytoplasmic signaling domain, a CD3 zeta cytoplasmic signaling domain, an OX40 cytoplasmic signaling domain, and/or a CD137 (4-1BB) cytoplasmic signaling domain.

33. The method of claim 1, wherein the cytoplasmic domain of the CAR comprises a CD3 zeta cytoplasmic signaling domain.

34. The method of claim 1, wherein the human subject having cancer has a cancer selected from the group consisting of leukemia, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphoblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

35. The method of claim 10, wherein the anti-CD2 ADC is administered to the patient as a single dose or as multiple doses.

36. The method of claim 10, wherein the anti-CD2 antibody, or antigen-binding fragment thereof, comprises a heavy chain variable region comprising a CDR1, a CDR2, and a CDR3 having an amino acid sequence as set forth in SEQ ID NOs: 1, 2, and 3, respectively, and comprises a light chain variable region comprising a CDR1, a CDR2, and a CDR3 having an amino acid sequence as set forth in SEQ ID NOs: 4, 5, and 6, respectively.

37. The method of claim 10, wherein the cytotoxin is an antimitotic agent or an RNA polymerase inhibitor.

38. The method of claim 37, wherein the RNA polymerase inhibitor is an amatoxin.

39. The method of claim 37, wherein the antimitotic agent is a maytansine, an auristatin. a pyrrolobenzodiazepine (PBD), or a calicheamicin.

40. The method of claim 10, wherein the extracellular domain of the CAR comprises an scFv antibody or a single chain T cell receptor (scTCR).

41. The method of claim 10, wherein the tumor antigen is an antigen selected from the group consisting of CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

42. The method of claim 10, wherein the cytoplasmic domain of the CAR comprises a CD28 cytoplasmic signaling domain, a CD3 zeta cytoplasmic signaling domain, an OX40 cytoplasmic signaling domain, and/or a CD137 (4-1BB) cytoplasmic signaling domain.

43. The method of claim 10, wherein the human subject having cancer has a cancer selected from the group consisting of leukemia, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphoblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.
